(19) United States
(12) Patent Application Publication
(10) Pub. No.: US 2007/0134341 A1
(43) Pub. Date: Jun. 14, 2007

(54) COMPOSITIONS OF LIPOXYGENASE INHIBITORS

(76) Inventors: James E. Kipp, Wauconda, IL (US); Jane Werling, Arlington Heights, IL (US); Pramod Gupta, Pittsford, NY (US); Rita Buresh, Lindenhurst, IL (US)

Correspondence Address: BAXTER HEALTHCARE CORPORATION, ONE BAXTER PARKWAY, DF2-2E, DEERFIELD, IL, 60015, US

(21) Appl. No.: 11/560,324

(22) Filed: Nov. 15, 2006

Related U.S. Application Data
(60) Provisional application No. 60/737,005, filed on Nov. 15, 2005.

(51) Int. Cl. A61K 31/381; A61K 9/14

(52) U.S. Cl. 424/489; 514/443

(57) ABSTRACT

Pharmaceutical compositions comprising particles of lipoxygenase inhibitor compounds having an effective average size of from about 10 nm to about 50 microns are provided. More particularly, pharmaceutical compositions of particle of a 5-lipoxygenase inhibitor compound having an effective average size of from about 50 nm to about 5 microns are provided. The pharmaceutical compositions are in the form of aqueous suspensions with the particle of the 5-lipoxygenase inhibitor compound present in concentrations of from about 5 to about 200 mg/ml. In addition, methods for making such pharmaceutical compositions are provided. In particular, microprecipitation and direct homogenization in the presence of at least one surfactant are disclosed for making the pharmaceutical compositions.

This application claims the benefit of U.S. Provisional Application Ser. No. 60/737,005 filed on Nov. 15, 2006.

### BACKGROUND OF THE INVENTION

The present invention is directed to compositions of lipoxygenase inhibitors, methods for making the same and methods for treating conditions mediated by lipoxygenase and/or leukotriene activity. In particular, the invention is directed to stable formulations containing small particles of 5- and/or 12-lipoxygenase inhibitors at therapeutically effective concentrations, methods for making the same and methods of treating conditions mediated by lipoxygenase and/or leukotriene activity with such formulations. A preferred embodiment of the invention is directed to stable suspensions, and stable dried suspensions, containing small particles of zileuton at therapeutically effective concentrations for parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal administration, methods for making suspensions and dried suspensions and methods of treating conditions mediated by lipoxygenase and/or leukotriene activity with suspensions and dried suspensions.

Lipoxygenase enzymes play an important role in various diseases such as asthma, rheumatoid arthritis, gout, psoriases, allergic rhinitis, Crohn's disease, respiratory distress syndrome, chronic obstructive pulmonary disease, acne, atherosclerosis, aortic aneurysm, sickle cell disease, acute lung injury, ischemia/reperfusion injury, nasal polyposis and/or inflammatory bowel disease among others. Accordingly, compounds which inhibit lipoxygenase activity are useful in the treatment and/or prevention of such diseases. U.S. Pat. Nos. 4,873,259, 4,992,464, and 5,250,565, which are incorporated herein by reference and made a part hereof, disclose certain lipoxygenase inhibitors, particularly 5- and/or 12-lipoxygenase inhibiting compounds, methods of making 5- and/or 12-lipoxygenase inhibiting compounds and pharmaceutical formulations of 5- and 12-lipoxygenase inhibitors. One such lipoxygenase inhibitor is commonly known as zileuton. A solid dosage form of 600 mg zileuton for oral administration is used as a treatment for asthma (ZYFLO® FILMTAB® tablets).

Zileuton has the following chemical structure:

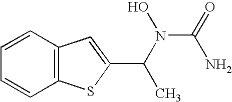

Zileuton may be used as a racemic mixture (about 50:50) of R(+) and S(−) enantiomers. Isomers of zileuton and their use in the inhibition of lipoxygenase activity have also been described. U.S. Pat. No. 5,629,337, which is incorporated herein by reference and made a part hereof, discloses the use of optically pure (−)-zileuton in the inhibition of lipoxygenase activity. WO 94/26268, which is incorporated herein by reference and made a part hereof, discloses the use of optically pure (+)-zileuton in the inhibition of lipoxygenase activity.

The poor solubility in water of some 5- and/or 12-lipoxygenase inhibitors prevents these beneficial agents from broader use than they would otherwise enjoy if aqueous formulations could be prepared at therapeutically effective concentrations for parenteral administration, particularly, formulations for injection. Zileuton for example is soluble in methanol and ethanol, slightly soluble in acetonitrile, and practically insoluble in hexane and water (water solubility 0.08-0.14 mg/ml at 25° C.) Trivedi, J. S. et al., Solubility and Stability Characterization of Zileuton in a Ternary Solvent System., European J. Pharm. Sci., 1996, volume 4, pages 109-116. In addition to its poor solubility, zileuton and likely other 5-lipoxygenase inhibitors of the N-hydroxyurea class may be chemically unstable in aqueous solution for storage at room temperature for prolonged periods of time. Alvarez, F J, Kinetics and Mechanism of Degradation of Zileuton, a Potent 5-Lipoxygenase Inhibitor., Pharm. Res., 1992, volume 9(11), pages 1465-1473.

The poor solubility in water of 5- and/or 12-lipoxygenase inhibitors presents a significant obstacle in providing these agents for parenteral administration, at least at therapeutically effective concentrations. Poorly soluble and insoluble compounds are, for example, compounds that have a solubility of 10 mg/ml or less in water. Although insoluble agents can be administered orally, oral bioavailability of highly water-insoluble drugs is often quite limited and variable, requiring the development of improved formulations.

Methods for modification of a poorly soluble or insoluble drug itself in an attempt to render it more suitable for parenteral administration include altering the morphology or molecular structure of the drug. In many instances, these methods have a number of shortcomings. For example, when modifying the morphology of the drug itself, the apparent solubility rather than the true solubility of the drug is altered, which may cause physical instability of the drug. Furthermore, although modifying the molecular structure of the drug itself alters true solubility of the drug, this requires extensive development time and clinical work in selecting a suitable molecular site for synthetic elaboration and in implementing the synthesis.

Other methods include vehicle modification of a poorly soluble or insoluble drug and include the use of salt formation, co-solvent/solubilization, solid carrier systems, micellization, lipid vesicle, oil-water partitioning, and complexation. Nevertheless, in many instances, these methods also have a number of shortcomings. For example, salt formation alters the pH of the drug; therefore, this method of delivery is limited by the intrinsic solubility of the drug, salt solubility, and pKa. The use of co-solvents is further limited by solvent choice and high osmolality. Furthermore, high solubility enhancement using co-solvents requires a substantial fraction of co-solvent which may increase the toxicity of the formulation.

Therefore, there is a need for compositions of 5- and/or 12-lipoxygenase inhibitors having a therapeutically effective concentration of the lipoxygenase inhibitor and that can be safely administered parenterally and/or orally, and in particular small particle compositions having therapeutically effective concentrations of a 5-lipoxygenase inhibitor for parenteral administration, for example by injection. Moreover, a need exists for small particle suspensions of 5- and/or 12-lipoxygenase inhibitors which can provide therapeutically effective concentrations that are stable and do not cause adverse effects from undesirably high concentrations of excipients.

One approach for delivering a poorly soluble or insoluble agent is to formulate the drug as a solid particle suspension. Drugs that are insoluble in water can provide the significant benefit of stability when formulated as a suspension of particles in an aqueous medium to create a microparticulate or nanoparticulate suspension. In this way, drugs that were previously unable to be formulated in an aqueous based system can be made suitable for intravenous administration. However, accurate control of particle size is essential for safe and efficacious use of these formulations.

Suspensions of solid particles having effective average size of from about 15 nm to about 1 micron are commonly referred to as nanosuspensions, and are most suitable for intravenous administration because their size range permits passage through the smallest blood vessels of the human circulatory system. These suspensions generally include small particles of insoluble compounds.

One approach to preparing a small-particle suspension is described in U.S. Pat. Nos. 6,607,784 and 6,951,656, which are incorporated herein by reference and made a part hereof. The '656 patent discloses a method for preparing submicron sized particles of an organic compound, wherein the solubility of the organic compound is greater in a water-miscible selected solvent than in another solvent which is aqueous. The process described in the '656 patent generally includes the steps of (i) dissolving the organic compound in the water-miscible selected solvent to form a first solution, (ii) mixing the first solution with a second solvent to precipitate the compound to define a pre-suspension; and (iii) adding energy to the pre-suspension to form particles which can be of submicron size. Often, the average effective particle size can range between about 100 nm to 1000 nm or below, extending into low micron size, typically no greater than about 2 microns.

Yet another attempt to provide insoluble drug formulations for parenteral delivery is disclosed in U.S. Pat. No. 5,922,355. The '355 patent discloses providing submicron sized particles of insoluble drugs using a combination of surface modifiers and a phospholipid, followed by particle size reduction using techniques such as sonication, homogenization, milling, microfluidization, precipitation or recrystallization. There is no disclosure in the '355 patent of changing process conditions to make crystals in a more friable form.

U.S. Pat. No. 5,858,410 discloses a pharmaceutical small-particle suspension suitable for parenteral administration. The '410 patent describes a method of subjecting at least one solid, therapeutically active compound dispersed in a solvent to high pressure homogenization in a piston-gap homogenizer. The particles formed have an average diameter, determined by photon correlation spectroscopy (PCS), of 10 nm to 1000 nm, and the proportion of particles larger than 5 microns in the total population being less than 0.1% (number distribution determined with a Coulter counter), without prior conversion into a melt. The examples in the '410 patent disclose jet milling prior to homogenization. Use of solvents is discouraged in that such use results in the formation of crystals that are too large.

Another approach to providing formulations of insoluble drugs for parenteral delivery is disclosed in U.S. Pat. No. 5,145,684. The '684 patent discloses the wet milling of an insoluble drug in the presence of a surface modifier to provide a drug particle having an average effective particle size of less than 400 nm. The surface modifier is adsorbed on the surface of the drug particle in an amount sufficient to prevent agglomeration into larger particles. The methods of the '684 patent, however, discourage the use of solvents to form precipitates in that such solvents may be very difficult to remove to pharmaceutically acceptable levels.

Forming small particle compositions of 5- and/or 12-lipoxygenase inhibitors such as zileuton can lead to increased therapeutic efficacy and increased therapeutic applications of the drug. For example, small particle suspensions having therapeutically effective concentrations of lipoxygenase inhibitors can be formulated into ready-to-use injectable compositions such as an I.V. push or bolus injection compositions. In addition, small particle suspensions can be prepared having higher concentrations of the lipoxygenase inhibitor for later dilution prior to injection. Injectable formulations of lipoxygenase inhibitors could permit its use in treating a broad array of conditions mediated by lipoxygenase and/or leukotriene activity.

Once small particle suspensions having therapeutically effective concentrations of lipoxygenase inhibitors have been prepared, solid concentrates can also be prepared by known methods, such as lyophilization, spray-drying and/or supercritical fluid extraction. These solid concentrates can then be resuspended at the time of injection. Also, these solid concentrates may also be compounded to produce a single dosage form such as tablets, capsules, lozenges, suppositories, coated tablets, capsules, ampoules, suppositories, delayed release formulations, controlled release formulations, extended release formulations, pulsatile release formulations, immediate release formulations, gastroretentive formulations, effervescent tablets, fast melt tablets, oral liquid and sprinkle formulations. The solid concentrates may also be formulated in a form selected from the group consisting of a patch, a powder preparation for inhalation, a suspension, an ointment and an emulsion.

Small particle compositions of 5- and/or 12-lipoxygenase inhibitors such as zileuton can also be formulated in therapeutically effective concentrations for delivery as an aerosol for respiratory delivery to the lungs, as a suspension for topical ophthalmic delivery or as a suspension for intranasal delivery.

### SUMMARY OF THE INVENTION

In one aspect of the present invention, there is provided a pharmaceutical composition comprising an aqueous suspension of particles of a lipoxygenase inhibitor compound, wherein the particles have an effective average size of from about 10 nm to about 50 microns.

In another aspect of the invention, the pharmaceutical composition comprises particles of a lipoxygenase inhibitor compound and at least one pharmaceutically acceptable excipient, wherein the particles have an effective average size from about 10 nm to about 50 microns and wherein the lipoxygenase inhibitor is present in a therapeutically effective amount.

In another aspect of the present invention, a method of treating a mammal suffering from a condition mediated by lipoxygenase and/or leukotriene activity by administering the pharmaceutical composition comprising an aqueous suspension of particles of a lipoxygenase inhibitor compound, wherein the particles have an effective average size of from about 10 nm to about 50 microns is provided.

In another aspect of the present invention, a method of making a pharmaceutical composition comprising particles of a lipoxygenase inhibitor compound having an effective average size of from about 10 nm to about 50 microns by homogenization is provided.

In another aspect of the present invention, a method of making a pharmaceutical composition comprising particles of a lipoxygenase inhibitor compound having an effective average size of from about 10 nm to about 50 microns by a microprecipitation method is provided.

In another aspect of the present invention, a method of making a pharmaceutical composition comprising particles of a lipoxygenase inhibitor compound having an effective average size of from about 10 nm to about 50 microns by a microprecipitation method with energy addition is provided.

In yet another aspect of the present invention, a method of making a pharmaceutical composition comprising particles of a lipoxygenase inhibitor compound having an effective average size of from about 10 nm to about 50 microns is provided. The method comprises dissolving the lipoxygenase inhibitor compound in a water-miscible solvent to form a solution; mixing the solution with another solvent to define a pre-suspension; and adding energy to the pre-suspension to form particles of the lipoxygenase inhibitor compound having an effective average particle size of from about 15 nm to about 50 microns.

### BRIEF DESCRIPTION OF THE DRAWINGS

FIG. 1 shows a flowchart of Method A of the microprecipitation method.

FIG. 2 shows a flowchart of Method B of the microprecipitation method.

FIG. 3 shows the comminution profile for Formulations A1 and A2.

FIG. 4 shows the comminution profile for Formulations B1 and B2.

FIG. 5 shows particle size measurements for Formulation A1 following stress testing.

FIG. 6 shows particle size measurements for Formulation A2 following stress testing.

FIG. 7 shows the dissolution of Formulation A1 in a solution of Sorensen's buffer and 5% w/v albumin over time.

FIG. 8 shows the dissolution of increasing doses of Formulation A1 in solution of Sorensen's buffer and 5% w/v albumin over time.

FIG. 9 shows particle size measurements for Formulation C following stress testing.

FIG. 10 shows particle size measurements for Formulation D following stress testing.

FIG. 11 shows particle size measurements for Formulation E following stress testing.

FIG. 12 shows particle size measurements for Formulation F following stress testing.

FIG. 13 shows particle size measurements for Formulation G following stress testing.

FIG. 14 shows particle size measurements for Formulation G following storage at 5° C.

FIG. 15 shows particle size measurements for Formulation G following storage at 25° C.

FIG. 16 shows particle size measurements for Formulations H, I, J, and K.

FIG. 17 shows particle size measurements for Formulation K following stress testing.

FIG. 18 shows the initial dissolution profile of lyophilized and non-lyophilized suspension of Formulation L.

### DETAILED DESCRIPTION OF THE INVENTION

As used herein, “a” or “an” are taken to mean one or more unless otherwise specified.

The present invention encompasses several different embodiments. Preferred embodiments of the invention are disclosed with the understanding that the present disclosure is to be considered as exemplifications of the principles of the invention and are not intended to limit the broad aspects of the invention to the embodiments illustrated.

The present invention is directed to small-particle suspensions of lipoxygenase inhibitors and preferably to 5- and/or 12-lipoxygenase inhibitors. Such lipoxygenase inhibitors are described for example in U.S. Pat. Nos. 4,873,259, 4,992,464, 5,250,565, 5,629,337 and WO 94/26268. Preferred 5- and/or 12-lipoxygenase inhibitors are of the type having the Formula (I):

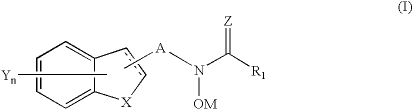

wherein R₁ is selected from the group consisting of hydrogen, C1-C4 alkyl, C2-C4 alkenyl, and NR₂R₃, wherein R₂ and R₃ are each independently selected from hydrogen, C1-C4 alkyl and hydroxyl, but R₂ and R₃ are not simultaneously hydroxyl;

wherein X is oxygen, sulfur, SO₂, or NR₄, wherein R₄ is selected from the group consisting of hydrogen, C1-C6 alkyl, C1-C6 alkoyl, aroyl and alkylsulfonyl;

A is selected from C1-C6 alkylene and C2-C6 alkenylene;

n is 1-5;

each Y is independently selected from hydrogen, halo, hydroxyl, cyano, halosubstituted alkyl, C1-C12 alkyl, C2-C12 alkenyl, C1-C12 alkoxy, C3-C8 cycloalkyl, C1-C8 thioalkyl, aryl, aryloxy, aroyl, C1-C12 arylalkyl, C2-C12 arylalkenyl, C1-C12 arylalkoxy and C1-C12 arylthioalkoxy, wherein substitutents are selected from halo, nitro, cyano, C1-C12 alkyl, alkoxy and halosubstituted alkyl;

Z is oxygen or sulfur; and

M is hydrogen, a pharmaceutically acceptable cation, aroyl or C1-C12 alkoyl.

The substituent(s) Y and the linking group A may be attached at any available position of either ring.

In an additional embodiment, the 5- and/or 12-lipoxygenase inhibitors are of the type having the Formula (II): 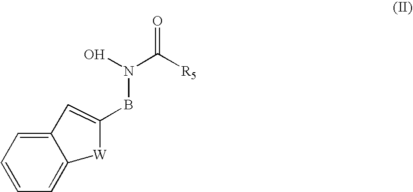
where R₅ is C₁ or C₂ alkyl, or NR₆R₇ where R₆ and R₇ are independently selected from hydrogen and C₁ or C₂ alkyl; B is CH₂ or CHCH₃; and W is oxygen, sulfur, or nitrogen.

The term “alkylene” is used herein to mean straight or branched chain spacer radicals, for example, —CH₂—, —C(CH₃)₂—, —CH(C₂H₅)—, —CH₂CH₂—, —CH₂CHCH₃—, —C(CH₃)₂—, C(CH₃)₂—, CH₂CH₂CH₂.

The term “alkenylene” is used herein to mean straight or branched chain unsaturated spacer radicals, for example, —CH═CH—, —CH═CHCH₂—, CH═CHCH(CH₃)—, —C(CH₃)═CHCH₂—, —CH₂CH═CHCH₂—, —C(CH₃)₂CH═CHC(CH₃)₂—.

The term “alkyl” is used herein to mean straight or branched chain radicals of 1 to 12 carbon atoms, including, but not limited to methyl, ethyl, n-propyl, isopropyl, n-butyl, sec-butyl, isobutyl and tert-butyl.

The term “alkenyl” is used herein to mean straight or branched chain unsaturated radicals of 2 to 12 carbon atoms, including, but not limited to ethenyl, 1-propenyl, 2-propenyl, 2-methyl-1-propenyl, 1-butenyl, 2-butenyl.

The term “cycloalkyl” is used herein to mean cyclic radicals, for example, of 3 to 8 carbons, including, but not limited to cyclopropyl, cyclobutyl, cyclopentyl and cyclohexyl.

The term “alkoxy” is used herein to mean —OR₈ wherein R₈ is an alkyl radical, including, but not limited to methoxy, ethoxy, isopropoxy, n-butoxy, sec-butoxy, isobutoxy, tert-butoxy, and the like.

The term “thioalkyl” is used herein to mean —SR₉ wherein R₉ is an alkyl radical, including, but not limited to thiomethyl, thioethyl, thioisopropyl, n-thiobutyl, sec-thiobutyl, isothiobutyl and tert-thiobutyl.

The term “alkoyl” is used herein to mean —COR₁₀ wherein R₁₀ is an alkyl radical, including, but not limited to formyl, acetyl, propionyl, butyryl, isobutyryl and pivaloyl.

The term “carboalkoxy” is used herein to mean —COR₁₁ wherein R₁₁ is an alkoxy radical, including, but not limited to carbomethoxy, carboethoxy, carboisopropoxy, carbobutoxy, carbosec-butoxy, carboiso- butoxy and carbotert-butoxy.

The term “aryl” is used herein to mean substituted and unsubstituted carbocyclic and heterocylic aromatic radicals wherein the substituents are chosen from halo, nitro, cyano, alkyl, alkoxy, and halosubstituted alkyl, including, but not limited to phenyl, 1- or 2-naphthyl, 2-, 3-, or 4-pyridyl, 2- and 3-furyl.

The term “aroyl” is used herein to mean —COR₁₂ wherein R₁₂ is an aryl radical, including, but not limited to benzoyl, 1-naphthoyl and 2-naphthoyl.

The term “aryloxy” is used herein to mean —OR₁₃ wherein R₁₃ is an aryl radical, including, but not limited to phenoxy, 1-naphthoxy and 2-naphthoxy.

The term “arylalkoxy” is used herein to mean —OR₁₄ wherein R₁₄ is an arylalkyl radical, including, but not limited to phenylmethoxy (i.e., benzyloxy), 4-fluorobenzyloxy, 1-phenylethoxy, 2-phenylethoxy, diphenylmethoxy, 1-naphthylmethoxy, 2-napthylmethoxy, 9-fluorenoxy, 2-, 3- or 4-pyridylmethoxy and 2-, 3-,4-, 5-, 6-, 7-, 8-quinolylmethoxy.

The term “arylthioalkoxy” is used herein to mean —SR₁₅ wherein R₁₅ is an arylalkyl radical, including, but not limited to phenylthiomethoxy (i.e., thiobenzyloxy), 4-fluorothiobenzyloxy, 1-phenylthioethoxy, 2-phenylthioethoxy, diphenylthiomethoxy and 1-naphthylthiomethoxy.

The term “arylalkyl” is used herein to mean an aryl group appended to an alkyl radical, including, but not limited to phenylmethyl (benzyl), 1-phenylethyl, 2-phenylethyl, 1-naphthylethyl and 2-pyridylmethyl.

The term “arylalkenyl” is used herein to mean an aryl group appended to an alkenyl radical, including, but not limited to phenylethenyl, 3-phenylprop-1-enyl, 3-phenylprop-2-enyl and 1-naphthylethenyl.

The term “alkylsulfonyl” is used herein to mean —SO₂R₁₆ wherein R₁₆ is an alkyl radical, including, but not limited to methylsulfonyl (i.e. mesityl), ethyl sulfonyl and isopropylsulfonyl.

The terms “halo” and “halogen” are used herein to mean radicals derived from the elements fluorine, chlorine, bromine, or iodine.

The term “halosubstituted alkyl” refers to an alkyl radical as described above substituted with one or more halogens, including, but not limited to chloromethyl, trifluoromethyl, 2,2,2-trichloroethyl, and the like.

The term “pharmaceutically acceptable cation” refers to non-toxic cations including but not limited to cations based on the alkali and alkaline earth metals, such as sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine and ethylamine.

One particular lipoxygenase inhibitor compound, zileuton, has been clinically approved for the treatment of asthma by oral administration. Accordingly, a preferred lipoxygenase inhibitor of the present invention is zileuton which has the Formula (III):

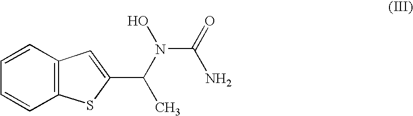

Certain of the lipoxygenase inhibitors described herein contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

As used, herein, the term “zileuton” encompasses ((±)-1-(1-benzo[b]thien-2-ylethyl)-1-hydroxyurea, the optically pure form of the (S)-enantiomer or (−)-isomer of 1-(1-benzo[b]thien-2-ylethyl)-1-hydroxyurea (described, for example, in U.S. Pat. No. 5,629,337), the optically pure form of (R)-enantiomer or (+)-isomer of N-(1-benzo[b]thien-2-ylethyl)-N-hydrxoyurea (described, for example, in WO 94/26268), mixtures of said (S)- and (R)-isomers in any ratio between 1:99 and 99:1, and polymorphic forms of zileuton, now known or later discovered.

In one embodiment, the lipoxygenase inhibitor compound is selected from the group consisting of ((±)-1-(1-benzo[b]thien-2-ylethyl)-1-hydroxyurea, the optically pure (−)-isomer of 1-(1-benzo[b]thien-2-ylethyl)-1-hydroxyurea and the optically pure (+)-isomer of 1-(1-benzo[b]thien-2-ylethyl)-1-hydroxyurea.

The present invention provides compositions of small particles of lipoxygenase inhibitors, methods for making small particles of lipoxygenase inhibitors and methods for treating conditions mediated by lipoxygenase and/or leukotriene activity with small particles of lipoxygenase inhibitors. The small particles of the lipoxygenase inhibitors of the present invention typically have an effective average particle size of from about 50 nm to about 10 microns, preferably from about 100 nm to about 5 microns, and more preferably from about 100 nm to about 2 microns as measured by methods including, but not limited to, light scattering (e.g., photocorrelation spectroscopy, laser diffraction, low-angle laser light scattering (LALLS), medium-angle laser light scattering (MALLS)), light obscuration (HIAC counter, for example), electrical resistance (Coulter method, for example), rheology, microscopy (light, electron or atomic-force, for example), or by fractionation such as gradient density centrifugation or force-field fractionation. The particles, however, can be prepared in a wide range of sizes, such as from about 10 nm to about 50 microns, preferably from about 20 nm to about 20 microns, more preferably from about 50 nm to about 2 microns. The preferred average effective particle size depends on factors such as the intended route of administration, formulation, dissolution rate, physical and chemical stability, solubility, toxicity and bioavailability of the compound.

Small particles of an insoluble compound can be made using any appropriate method including, but not limited to, precipitation methods, mechanical/physical particle size reduction methods such as milling and homogenization, phospholipids coating methods, HLB surfactant coating methods, spray-drying methods, supercritical fluid methods, and hot melt methods, such as those disclosed in U.S. Pat. Nos. 2,745,785, 5,118,528, 4,826,689, 5,091,188; 5,091,187, 4,725,442, 5,145,684, 5,780,062, 5,858,410, 4,997,454, 6,604,698, 6,634,576, 6,682,761, 5,922,355, 6,337,092, 6,387,409, 6,177,103, 6,835,396, 6,869,617, 6,884,436, Re. 35,338, 5,560,932, 5,662,883, 5,665,331, 5,510,118, 5,518,187, 5,534,270 5,718,388, 5,862,999, 5,605,785, 5,665,331, U.S. Pre-grant publication nos. U.S. 2002/003179, 2004/0164194, 2004/0173696, PCT publication nos. WO01/21154, WO00/30615, and commonly assigned and co-pending U.S. patent applications Ser. Nos. 09/874,499, 09/964,273, 10/035,821, 10/213,352, 10/246,802, 10/270,268, 10/270,267, 10/390,333, 10/696,384 (U.S. Patent publication No. 2004/02567), 10/806,050, 10/920,578, 10/703,395, 11/052276, and 11/224,633 which are incorporated herein by reference and made a part hereof. Preferred methods of making small particles of a lipoxygenase inhibitor are methods involving microprecipitation and energy addition such as those disclosed in the '656 patent and direct homogenization methods similar to methods disclosed in the '410 patent. A general procedure for both preferred methods of preparing the small particle compositions of the present invention follows.

Precipitation

The processes can be separated into four general categories. Each of the categories of processes share the steps of: (1) dissolving lipoxygenase inhibitor in a water miscible organic solvent to create a first solution; (2) mixing the first solution with a second solution that contains water, to precipitate the lipoxygenase inhibitor to create a pre-suspension; and, optionally, (3) adding energy to the pre-suspension in the form of high-shear mixing or heat to provide a stable form of the lipoxygenase inhibitor having the desired size ranges defined above.

The four categories of processes can be distinguished based upon the physical properties of the precipitate, for example, as determined through x-ray diffraction studies, differential scanning calorimetry (DSC) studies or other suitable study conducted prior to the energy-addition step and after the energy-addition step.

First Process Category

The methods of the first process category generally include the step of dissolving the lipoxygenase inhibitor in a water miscible first solvent followed by the step of mixing this solution with an aqueous solution to form a pre-suspension wherein the lipoxygenase inhibitor is in an amorphous form, a semi-crystalline form or in a supercooled liquid form as determined by x-ray diffraction, DSC, light or electron microscopy or other analytical techniques and has an average effective particle size within one of the effective particle size ranges set forth above. The mixing step is followed by an energy-addition step and, in a preferred form of the invention is an annealing step (see the '656 patent).

Second Process Category

The methods of the second process category include essentially the same steps as in the steps of the first process category but differ in the following respect. An x-ray diffraction, DSC or other suitable analysis of the pre-suspension shows the lipoxygenase inhibitor in a crystalline form and having an average effective particle size. The lipoxygenase inhibitor compound after the energy-addition step has essentially the same average effective particle size as prior to the energy-addition step but has less of a tendency to aggregate into larger particles when compared to that of the particles of the pre-suspension. Without being bound to a theory, it is believed the differences in the particle stability may be due to a reordering of the surfactant molecules at the solid-liquid interface.

Third Process Category

The methods of the third category modify the first two steps of those of the first and second processes categories to ensure the lipoxygenase inhibitor in the pre-suspension is in a friable form having an average effective particle size (e.g., such as slender needles and thin plates). Friable particles can be formed by selecting suitable solvents, surfactants or combination of surfactants, the temperature of the individual solutions, the rate of mixing and rate of precipitation and the like. Friability may also be enhanced by the introduction of lattice defects (e.g., cleavage planes) during the steps of mixing the first solution with the aqueous solution. This would arise by rapid crystallization such as that afforded in the precipitation step. In the energy-addition step these friable crystals are converted to crystals that are kinetically stabilized and having an average effective particle size smaller than those of the presuspension. Kinetically stabilized means particles have a reduced tendency to aggregate when compared to particles that are not kinetically stabilized. In such instance the energy-addition step results in a breaking up and coating of the friable particles. By ensuring the particles of the presuspension are in a friable state, the organic compound can more easily and more quickly be prepared into a particle within the desired size ranges when compared to processing an organic compound where the steps have not been taken to render it in a friable form.

Fourth Process Category

In the fourth process category, the first solution and second solvent are simultaneously subjected to the energy-addition step. Thus, friable material is generated in-situ and immediately comminuted as it is created.

The energy-addition step can be carried out in any fashion wherein the pre-suspension is exposed to cavitation, turbulence, pressure gradient, shearing or impact forces. In one preferred form of the invention, the energy-addition step is an annealing step. Annealing is defined in this invention as the process of converting matter that is thermodynamically unstable into a more stable form by single or repeated application of energy (direct heat or mechanical stress), followed by relaxation. This lowering of energy may be achieved by conversion of the solid form from a less ordered to a more ordered lattice structure. Alternatively, this stabilization may occur by a reordering of the surfactant molecules at the solid-liquid interface.

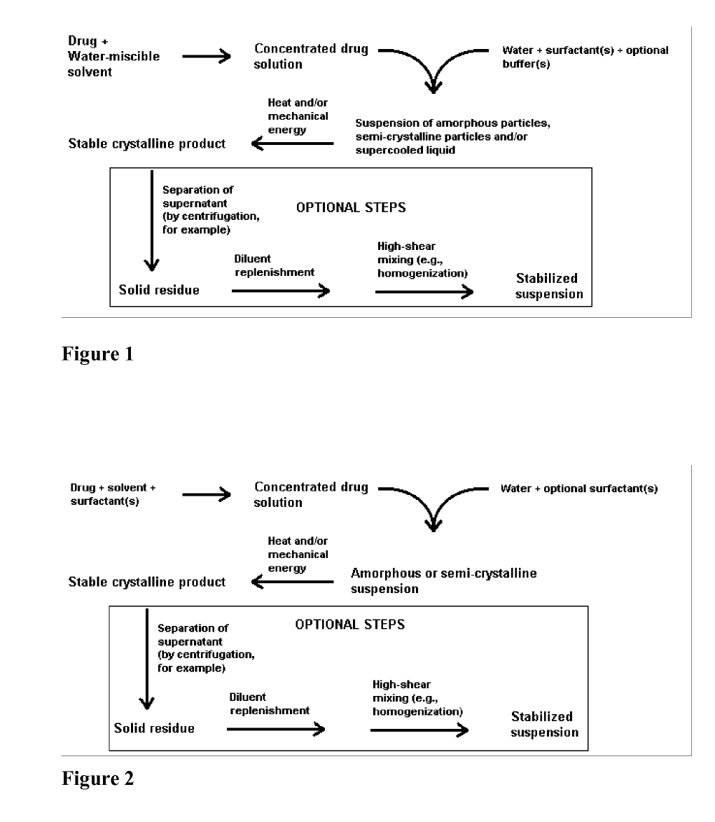

The first process category, as well as the second, third, and fourth process categories, can be further divided into two subcategories, Method A and B shown diagrammatically in FIG. 1 and FIG. 2, respectively.

The first solvent according to the present invention is a solvent or mixture of solvents in which the organic compound of interest is relatively soluble and which is miscible with the second solvent. Such solvents include, but are not limited to water-miscible protic compounds, in which a hydrogen atom in the molecule is bound to an electronegative atom such as oxygen, nitrogen, or other Group VA, VIA and VII A in the Periodic Table of elements. Examples of such solvents include, but are not limited to, alcohols, amines (primary or secondary), oximes, hydroxamic acids, carboxylic acids, sulfonic acids, phosphonic acids, phosphoric acids, amides and ureas.

Other examples of the first solvent also include aprotic organic solvents. Some of these aprotic solvents can form hydrogen bonds with water, but can only act as proton acceptors because they lack effective proton donating groups. One class of aprotic solvents is a dipolar aprotic solvent, as defined by the International Union of Pure and Applied Chemistry (IUPAC Compendium of Chemical Terminology, 2nd Ed., 1997):
A solvent with a comparatively high relative permittivity (or dielectric constant), greater than ca. 15, and a sizable permanent dipole moment, that cannot donate suitably labile hydrogen atoms to form strong hydrogen bonds, e.g. dimethyl sulfoxide.

Dipolar aprotic solvents can be selected from the group consisting of: amides (fully substituted, with nitrogen lacking attached hydrogen atoms), ureas (fully substituted, with no hydrogen atoms attached to nitrogen), ethers, cyclic ethers, nitriles, ketones, sulfones, sulfoxides, fully substituted phosphates, phosphonate esters, phosphoramides, nitro compounds, and the like. Dimethylsulfoxide (DMSO), N-methyl-2-pyrrolidinone (NMP), 2-pyrrolidinone, 1,3-dimethylimidazolidinone (DMI), dimethylacetamide (DMA), dimethylformamide (DMF), dioxane, acetone, tetrahydrofuran (THF), tetramethylenesulfone (sulfolane), acetonitrile, and hexamethylphosphoramide (HMPA), nitromethane, among others, are members of this class.

Solvents may also be chosen that are generally water-immiscible, but have sufficient water solubility at low volumes (less than 10% v/v) to act as a water-miscible first solvent at these reduced volumes. Examples include aromatic hydrocarbons, alkenes, alkanes, and halogenated aromatics, halogenated alkenes and halogenated alkanes. Aromatics include, but are not limited to, benzene (substituted or unsubstituted), and monocyclic or polycyclic arenes. Examples of substituted benzenes include, but are not limited to, xylenes (ortho, meta, or para), and toluene. Examples of alkanes include but are not limited to hexane, neopentane, heptane, isooctane, and cyclohexane. Examples of halogenated aromatics include, but are not restricted to, chlorobenzene, bromobenzene, and chlorotoluene. Examples of halogenated alkanes and alkenes include, but are not restricted to, trichloroethane, methylene chloride, ethylenedichloride (EDC), and the like.

Examples of the all of the above solvent classes include but are not limited to: N-methyl-2-pyrrolidinone (N-methyl-2-pyrrolidone), 2-pyrrolidinone (2-pyrrolidone), 1,3-dimethyl-2-imidazolidinone (DMI), dimethylsulfoxide, dimethylacetamide, carboxylic acids (such as acetic acid and lactic acid), aliphatic alcohols (such as methanol, ethanol, isopropanol, 3-pentanol, and n-propanol), benzyl alcohol, glycerol, butylene glycol (1,2-butanediol, 1,3-butanediol, 1,4-butanediol, and 2,3-butanediol), ethylene glycol, propylene glycol, mono- and diacylated glycerides, dimethyl isosorbide, acetone, dimethylsulfone, dimethylformamide, 1,4-dioxane, tetramethylenesulfone (sulfolane), acetonitrile, nitromethane, tetramethylurea, hexamethylphosphoramide (HMPA), tetrahydrofuran (THF), diethylether, tert-butylmethyl ether (TBME), aromatic hydrocarbons, alkenes, alkanes, halogenated aromatics, halogenated alkenes, halogenated alkanes, xylene, toluene, benzene, substituted benzene, ethyl acetate, methyl acetate, butyl acetate, chlorobenzene, bromobenzene, chlorotoluene, trichloroethane, methylene chloride, ethylenedichloride (EDC), hexane, neopentane, heptane, isooctane, cyclohexane, polyethylene glycol (PEG), PEG esters, PEG-4, PEG-8, PEG-9, PEG-12, PEG-14, PEG-16, PEG-120, PEG-75, PEG-150, polyethylene glycol esters, PEG-4 dilaurate, PEG-20 dilaurate, PEG-6 isostearate, PEG-8 palmitostearate, PEG-150 palmitostearate, polyethylene glycol sorbitans, PEG-20 sorbitan isostearate, polyethylene glycol monoalkyl ethers, PEG-3 dimethyl ether, PEG-4 dimethyl ether, polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate, and glycofurol (tetrahydrofurfuryl alcohol polyethylene glycol ether).

A preferred first solvent is N-methyl-2-pyrrolidinone (NMP). Other preferred first solvents are methanol and lactic acid.

The second solvent is an aqueous solvent. This aqueous solvent may be water by itself This solvent may also contain buffers, salts, surfactant(s), water-soluble polymers, preservatives, antimicrobials, antioxidants, cryo-protectants, wetting agents, viscosity agents, tonicity modifying agents, levigating agents, absorption enhancers, penetration enhancers, pH modifying agents, muco-adhesive agents, coloring agents, flavoring agents, diluting agents, emulsifying agents, suspending agents, solvents, co-solvents, buffers, and combinations of these excipients.

Suitable surfactants for coating, adhering or associating to the particles in the present invention can be selected from ionic surfactants, nonionic surfactants, zwitterionic surfactants, polymeric surfactants, phospholipids, biologically derived surfactants, amino acids and their derivatives or derivatives, combinations or conjugates of the surfactants described above. Ionic surfactants can be anionic or cationic. The surfactants are present in the compositions in an amount of from about 0.01% to 10% w/v, and preferably from about 0.05% to about 5% w/v.

Suitable anionic surfactants include but are not limited to: alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, calcium stearate, phosphates, sodium dodecylsulfate, carboxymethylcellulose calcium, carboxymethylcellulose sodium, dioctylsulfosuccinate, dialkylesters of sodium sulfosuccinic acid, sodium lauryl sulfate and phospholipids.

Suitable cationic surfactants include but are not limited to: quaternary ammonium compounds, benzalkonium chloride, cetyltrimethylammonium bromide, chitosans, lauryldimethylbenzylammonium chloride, acyl camitine hydrochlorides, alkyl pyridinium halides, cetyl pyridinium chloride, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15 trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, “POLYQUAT 10” (a mixture of polymeric quarternary ammonium compounds), tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, “MIRAPOL” (polyquatemium-2) “Alkaquat” (alkyl dimethyl benzylammonium chloride, produced by Rhodia), alkyl pyridinium salts, amines, amine salts, imide azolinium salts, protonated quaternary acrylamides, methylated quaternary polymers, and cationic guar gum. benzalkonium chloride, dodecyl trimethyl ammonium bromide, triethanolamine, and poloxamines.

Suitable nonionic surfactants include but are not limited to: polyoxyethylene fatty alcohol ethers, polyoxyethylene sorbitan fatty acid esters, alkyl polyoxyethylene sulfates, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, p-isononylphenoxypoly(glycidol), decanoyl-N-methylglucamide, n-decyl-β-D-glucopyranoside, n-decyl -β-D-maltopyranoside, n-dodecyl -β-D-glucopyranoside, n-dodecyl-β-D-maltoside, heptanoyl-N-methylglucamide, n-heptyl-β-D-glucopy-ranoside, n-heptyl-β-D-thioglucoside, n-hexyl-β-D-glucopyranosid-e; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof.

Suitable polymeric surfactants include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate), poly vinyl chloride polystyrene and polyvinylpryrrolidone.

Suitable biologically derived surfactants include, but are not limited to: lipoproteins, gelatin, casein, lysozyme, albumin, casein, heparin, hirudin, or other proteins.

A preferred ionic surfactant is a bile salt, and a preferred bile salt is deoxycholate. A preferred nonionic surfactant is a polyalkoxyether, and preferred polyalkoxyethers (polyoxyethylene-polypropylene block copolymers) are Poloxamer 188 and Poloxamer 407. Another preferred surfactant is a pegylated lipid, preferably a pegylated phospholipid.

In a preferred embodiment of the present invention, the particles are suspended in an aqueous medium further including a pH adjusting agent. Suitable pH adjusting agents include, but are not limited to, sodium hydroxide, hydrochloric acid, tris buffer, mono-, di-, tricarboxylic acids and their salts, citrate buffer, phosphate buffer, glycerol-1-phosphate, glycercol-2-phosphate, acetate, lactate, tris(hydroxymethyl)aminomethane, aminosaccharides, mono-, di- and trialkylated amines, meglumine (N-methylglucosamine), and amino acids.

The aqueous medium may additionally include an osmotic pressure adjusting agent, such as but not limited to glycerin, inorganic salts, a monosaccharide such as dextrose, a disaccharide such as sucrose, trehalose and maltose, a trisaccharide such as raffinose, and sugar alcohols such as mannitol and sorbitol.

Method A

In Method A, the lipoxygenase inhibitor is first dissolved in the first solvent to create a first solution. The lipoxygenase inhibitor can be added from about 0.01% to about 90% weight to volume (w/v) depending on the solubility of the lipoxygenase inhibitor in the first solvent, preferably methanol or N-methyl-2-pyrrolidinone. In one embodiment, the lipoxygenase inhibitor is added from about 0.01 to about 50% (w/v). In another embodiment, the lipoxygenase inhibitor is added from about 0.01 to about 20% (w/v). Heating of the concentrate from about 30° C. to about 100° C. may be necessary to ensure total dissolution of the lipoxygenase inhibitor in the first solvent.

A second aqueous solution is provided with one or more surfactants added thereto. The surfactant or surfactants can be selected from an ionic surfactant, a nonionic surfactant, a cationic surfactant, an anionic surfactant, a zwitterionic surfactant, a polymeric surfactant, a phospholipid, a biologically derived surfactant, amino-acid surfactants, derivatives of amino acid surfactants or derivatives, combinations or conjugates of the surfactants described above.

A preferred ionic surfactant is a bile salt, and a preferred bile salt is deoxycholate. Preferred nonionic surfactants are a polyalkoxyether and a polyoxyethylene. Preferred polyalkoxyethers (polyhatidyloxyethylene-polypropylene block copolymers) are Poloxamer 188 and Poloxamer 407 and preferred polyoxyethylenes are polysorbates such as Tween 80, and PEG fatty acid esters such as Solutol. Another preferred surfactant is a pegylated lipid, preferably a pegylated phospholipid such as mPEG-DSPE2000. Another preferred phospholipid is a mixture of purified egg lecithins, Lipoid E80 (produced by Lipoid LLC). More than one surfactant can be used. Preferred surfactant combinations are Poloxamer 188/deoxycholate, poloxamer 188/mPEG-DSPE(2000), Lipoid 80/mPEG-DSPE(2000), Tween 80/Poloxamer 188, phosphatidylglycerol/poloxamer 188, and phosphatidylglycerol/phosphatidic acid.

In a preferred embodiment of the present invention, the second aqueous solution further includes a pH adjusting agent. Suitable pH adjusting agents include, but are not limited to, sodium hydroxide, hydrochloric acid, tris buffer, mono-, di-, tricarboxylic acids and their salts, citrate buffer, phosphate buffer, glycerol-1-phosphate, glycercol-2-phosphate, acetate, lactate, tris(hydroxymethyl)aminomethane, aminosaccharides, mono-, di- and trialkylated amines, meglumine (N-methylglucosamine), succinate, benzoate, tartrate, carbonate and amino acids.

The second aqueous solution preferably includes an osmotic pressure adjusting agent, such as but not limited to glycerin, inorganic salts, a monosaccharide such as dextrose, a disaccharide such as sucrose, trehalose and maltose, a trisaccharide such as raffinose, and sugar alcohols such as mannitol and sorbitol.

The first and second solutions are then combined. Preferably, the first solution is added to the second solution at a controlled rate. The addition rate is dependent on the batch size, and precipitation kinetics for the lipoxygenase inhibitor. Typically, for a small-scale laboratory process (preparation of 1 liter), the addition rate is from about 0.05 cc per minute to about 50 cc per minute. During the addition, the solutions should be under constant agitation. It has been observed using light microscopy that amorphous particles, semi-crystalline solids, or a super-cooled liquid are formed to create a pre-suspension. The method further includes the step of subjecting the pre-suspension to an annealing step to convert the amorphous particles, super-cooled liquid or semi-crystalline solid to a crystalline more stable solid state. The resulting particles will have an average effective particle size as measured by methods including, but not limited to, light scattering methods (e.g., photocorrelation spectroscopy, laser diffraction, low-angle laser light scattering (LALLS), medium-angle laser light scattering (MALLS)), light obscuration methods (HIAC method, for example), electrical resistance methods (Coulter counter, for example), rheology, microscopy (light, electron, or atomic-force), or fractionation methods, within the ranges set forth above.

The energy-addition step involves adding energy through sonication, homogenization, countercurrent flow homogenization (e.g., the Mini DeBEE 2000 homogenizer, available from BEE Incorporated, NC, in which a jet of fluid is directed along a first path, and a structure is interposed in the first path to cause the fluid to be redirected in a controlled flow path along a new path to cause emulsification or mixing of the fluid), microfluidization, or other methods of providing impact, shear, turbulence, pressure gradient, or cavitation forces. The sample may be cooled or heated during this stage. In one preferred form of the invention the annealing step is effected by homogenization. In another preferred form of the invention the annealing may be accomplished by ultrasonication. In yet another preferred form of the invention the annealing may be accomplished by use of an emulsification apparatus as described in U.S. Pat. No. 5,720,551, incorporated herein by reference and made a part hereof.

Depending upon the rate of annealing, it may be desirable to adjust the temperature of the processed sample to within the range of from approximately 0° C. to 30° C. Alternatively, in order to effect a desired phase change in the processed solid, it may also be necessary to adjust the temperature of the pre-suspension to a temperature within the range of from about −80° C. to about 100° C. during the annealing step.

Method B

Method B differs from Method A in the following respects. The primary difference is that a surfactant or combination of surfactants is added to the first solution. The surfactant or surfactants may be selected from ionic surfactants, nonionic surfactants, cationic surfactants, anionic surfactants, zwitterionic surfactants, polymeric surfactants, phospholipids, biologically derived surfactants, amino-acid surfactants, derivatives of amino acid surfactants and derivatives, combinations or conjugates of those set forth above.

A preferred method of preparing the small particles of a lipoxygenase inhibitor consists of: (i) mixing into the water-miscible first solvent or the second solvent, or both the water-miscible first solvent and the second solvent a surface modifier or combination of modifiers, at least one of which comprising a polyoxyalkylether (e.g., Poloxamer 188) or a phospholipid conjugated with a water-soluble or hydrophilic polymer; (ii) dissolving the lipoxygenase inhibitor in the water-miscible first solvent to form a solution; (iii) mixing the solution with the second solvent to define a pre-suspension of particles; and (iv) homogenizing the pre-suspension to form a suspension of particles having an average effective particle size of no greater than about 2 microns.

A preferred water-miscible first solvent is N-methyl-2-pyrrolidinone or methanol.

The phospholipid used can be natural or synthetic. Examples of suitable phohospholipds include, but are not limited to, phosphatidylcholine, phosphatidylethanolamine, diacyl-glycero-phosphoethanolamine, phosphatidylserine, phosphatidylinositol, phosphatidylglycerol, phosphatidic acid, lysophospholipids, egg or soybean phospholipid or a combination thereof. The diacyl-glycero-phosphethanolamine can be selected from: dimyristoyl-glycero-phosphoethanolamine (DMPE), dipalmitoyl-glycero-phosphoethanolamine (DPPE), distearoyl-glycero-phosph-oethanolamine (DSPE), dioleolyl-glycero-phosphoethanolamine (DOPE) or the like.

In a preferred embodiment, the water-soluble or hydrophilic polymer conjugating to the phospholipid is polyethylene glycol (PEG), such as, but are not limited to, PEG 350, PEG 550, PEG 750, PEG 1000, PEG 2000, PEG 3000, and PEG 5000. Other hydrophilic polymer conjugates can also be used, e.g., dextran, hydroxypropyl methacrylate (HPMA), polyglutamate and the like.

Optionally, a second surface modifier can be mixed into the water-miscible first solvent or the second solvent or both the water-miscible first solvent and the second solvent. The second surface modifier may be needed to further stabilize the particles. The second surface modifier can be selected from anionic surfactants, cationic surfactants, nonionic surfactants, zwitterionic surfactants, polymeric surfactants and surface active biological modifiers as described above. A preferred second surface modifier is poloxamer, such as poloxamer 188.

More than one surfactant can be used. Preferred surfactant combinations are Poloxamer 188/deoxycholate, poloxamer 188/mPEG-DSPE(2000), Lipoid 80/mPEG-DSPE(2000), Tween 80/Poloxamer 188, phosphatidylglycerol/poloxamer 188, and phosphatidylglycerol/phosphatidic acid.

The size of the particles produced also can be controlled by the temperature at which the homogenization is carried out. In one embodiment, the homogenization is carried out at about 30° C. or greater, such as at about 40° C. or about 70° C.

A drug suspension resulting from application of the processes described in this invention may be administered directly as a ready to use injectable solution, provided that an appropriate means for solution sterilization is applied. In one embodiment a solvent-free small-particle suspension may be produced by solvent removal after precipitation. This can be accomplished by centrifugation, dialysis, diafiltration, force-field fractionation, high-pressure filtration or other separation techniques well known in the art. Removal of organic solvent is typically carried out by one to three successive centrifugation cycles; after each centrifugation the supernatant is decanted and discarded. A fresh volume of the suspension vehicle without the organic solvent is added to the remaining solids and the mixture is dispersed by homogenization. It will be recognized by others skilled in the art that other high-shear mixing techniques could be applied in this reconstitution step. In a preferred embodiment, the water-miscible first solvent is removed simultaneously with homogenization as described in detail in a co-pending and commonly assigned U.S. Patent Application Publication 2004/0256749A1.

Optionally, a solvent-free suspension may be produced by solvent removal after precipitation. This can be accomplished by centrifugation, dialysis, diafiltration, force-field fractionation, high-pressure filtration or other separation techniques well known in the art. Removal of organic solvent is typically carried out by one to three successive centrifugation cycles; after each centrifugation the supernatant is decanted and discarded. A fresh volume of the suspension vehicle without the organic solvent is added to the remaining solids and the mixture is dispersed by homogenization. It will be recognized by others skilled in the art that other high-shear mixing techniques could be applied in this reconstitution step.

Furthermore, any undesired excipients such as surfactants may be replaced by a more desirable excipient by use of the separation methods described in the above paragraph. The solvent and first excipient may be discarded with the supernatant after centrifugation or filtration. A fresh volume of the suspension vehicle without the solvent and without the first excipient may then be added. Alternatively, a new surfactant may be added. For example, a suspension consisting of drug, N-methyl-2-pyrrolidinone (solvent), Poloxamer 188 (first excipient), sodium deoxycholate, glycerol and water may be replaced with phospholipids (new surfactant), glycerol and water after centrifugation and removal of the supernatant.

Small-Particle Suspensions Using Direct Homogenization

The preparation of small-particle suspensions by direct homogenization is accomplished by adding the insoluble lipoxygenase inhibitor compound in an aqueous solution to form a presuspension. The presuspension is then homogenized until the desired particle size is obtained. However, as those skilled in the art understand, the particle will not continue to reduce in size indefinitely.

A piston gap homogenizer is a preferred device. Piston gap homogenizers are widely used in food production. During homogenization, the substance, usually an emulsion or suspension, is pressurized and then forced through a narrow gap. The high velocity attained in the gap lowers the pressure, at which point cavitation occurs. Upon exiting the gap, the vapor bubbles encounter a higher pressure environment and collapse or implode with great force, causing break-up of the particles or droplets in the suspension or emulsion. Other forces in the homogenizer thought to contribute to break-up include turbulence, shear, and impact forces. Since the gap of the homogenizer is very narrow e.g. about 25 microns, the presuspension of the drug is preferably made using a starting material having a particle size of about 25 microns. Other homogenization devices may also be employed such as the homogenizers manufactured by BEE International, Inc. (South Easton, Mass., USA).

Preferably, the aqueous presuspension includes at least one surfactant. Suitable surfactants can be selected from ionic surfactants, nonionic surfactants, zwitterionic surfactants, polymeric surfactants, phospholipids, biologically derived surfactants or amino acid surfactants and their derivatives. Ionic surfactants can be anionic or cationic. The surfactants are present in the presuspension in an amount of from about 0.01% to 10% w/v, and preferably from about 0.05% to about 3% w/v. The entire list of surfactants and preferred surfactants are the same as those identified in the microprecipitation method above.

It is also preferred that the aqueous medium further include a pH adjusting agent. Suitable pH adjusting agents include, but are not limited to, sodium hydroxide, hydrochloric acid, tris buffer, mono-, di-, tricarboxylic acids and their salts, citrate buffer, phosphate buffer, glycerol-1-phosphate, glycercol-2-phosphate, acetate, lactate, tris(hydroxymethyl)aminomethane, aminosaccharides, mono-, di- and trialkylated amines, meglumine (N-methylglucosamine), and amino acids. Preferred pH adjusting agents are selected from tris, citrate and phosphate buffers.

The aqueous medium may additionally include an osmotic pressure adjusting agent, such as but not limited to glycerin, inorganic salts, a monosaccharide such as dextrose, a disaccharide such as sucrose, trehalose and maltose, a trisaccharide such as raffinose, and sugar alcohols such as mannitol and sorbitol. Preferred osmotic adjusting agents are glycerin, sucrose and trehalose.

The lipoxygenase inhibitor can be added before or after the addition of pH and/or osmotic adjusting agents. Optionally, the lipoxygenase inhibitor may be jet-milled prior to processing with the homogenizer. The presuspension is then processed with a piston gap homogenizer. A typical piston-gap homogenizer is one manufactured by Avestin Inc., including the Emulsiflex(R) series of piston-gap homogenizers. The number of passes through the homogenizer can vary from 1 to about 2000.

Following either the microprecipitation or direct homogenization method, the liquid phase of the suspension can be removed to form a dry powder of the small particles. This can be accomplished by several methods, for example, lyophilization, spray-drying and super-critical fluid extraction. A preferred method is by lyophilization (freeze-drying) to form a lyophilized suspension for reconstitution into a suspension suitable for administration. For the purpose of preparing a stablized, dry solid, cryo-protectant and/or bulking agents such as polyvinylpyrrolidone (PVP), mannitol, sorbitol, sucrose, starch, lactose, trehalose or raffinose alone or in combination may be added prior to lyophilization. A preferred cryo-protectant is PVP which is added prior to lyophilization at about 0.05 to about 1.0% (w/v), more preferably at about 0.2 to about 0.5% (w/v).

The dry powder of particles can be provided as is to the healthcare provider where it can be resuspended in an appropriate diluent, such as a diluent suitable for parenteral, oral, ophthalmic, nasal, or buccal administration among others. The dry powder can be administered to a subject by the pulmonary route. The dry powder can be processed for administration to a subject by various routes, such as, but are not limited to, parenteral (including, for example, intravenous, intramuscular and subcutaneous), oral, pulmonary, aural, topical, ophthalmic, nasal, buccal, rectal, vaginal, intracerebral, intraocular, intradermal, intralymphatic, intraarticular, intrathecal, intraperitoneal and transdermal.

In addition, the dry powder can be re-suspended to produce ready-to-use formulations, which can then be provided to a healthcare provider. Ready-to-use injectable formulations can be prepared in high concentration dosages for direct administration or for further dilution by the health care provider. In a preferred embodiment, the small particles of the lipoxygenase inhibitor are suspended in an aqueous solution at a concentration of from about 0.1 to about 500 mg/ml, more preferably at a concentration of from about 1 to about 100 mg/ml and most preferably at a concentration of from about 10 to about 50 mg/ml.

In particular situations, providing lyophilized suspensions may be more desirable than providing aqueous suspensions because certain lipoxygenase inhibitor compounds may be chemically unstable in aqueous solutions in suspension form. This may be especially true if the suspensions will be subjected to harsh conditions such as extended transportation or storage in areas that experience extreme temperature fluctuations.

In another preferred embodiment, the small particles of the lipoxygenase inhibitor are physically stable i.e. do not aggregate under stressed conditions or upon storage. Stress testing methods for particles are well known in the art. Typical stress testing methods are described in detail in “Novel Injectable Formulations of Insoluble Drugs,” Pace et al., Pharm Tech, March 1999, pg 116-134. Examples of stressed conditions include, but are not limited to, thermal cycling, repeated freeze-thaw cycling, agitation, and centrifugation. Experimental data showed that the small particles of the lipoxygenase inhibitor remained stable after being subjected to freeze-thaw cycling, agitation, and centrifugation. Testing also indicated that the small-particle suspension remained physically stable after short term storage when stored at near freezing temperatures as well as at room temperature.

In another preferred embodiment, the compositions of small particles of the present invention are prepared in frozen form. The frozen form can withstand longer shelf-life and then be thawed prior to administration.

In another preferred embodiment, the small particles of the lipoxygenase inhibitor are suspended in an aqueous solution at a concentration of at least about 30 mg/ml and have a rapid drug release following in vivo injection such that the time for peak plasma concentration is reached within less than about 8 hours, more preferably within about 4 hours and most preferably within about 2 hours after dosing.

Sterilization can be accomplished in a number of methods. Methods for sterilizing pharmaceutical compositions include, but are not limited to filtration, heat sterilization, high-pressure sterilization and irradiation. Heat sterilization may be effected by the heat within the homogenizer, in which the homogenizer serves as a heating and pressurization source for sterilization. Further processing would require aseptic operating procedures. High-pressure sterilization of suspension formulations can be performed according to methods disclosed in commonly assigned U.S. patent application Ser. No. 10/946,885 (U.S. Patent Publication No. 2005/0135963), filed Sep. 22, 2004, incorporated herein by reference. A sterile composition may also be prepared with sterile starting material that can be added to the process stream aseptically.

In the precipitation methods, sterilization may be accomplished by separate sterilization of the drug concentrate (drug, solvent, and optional surfactant) and the diluent medium (water, and optional buffers and surfactants) prior to mixing to form the pre-suspension. Sterilization methods would include pre-filtration through a series of filters, followed by other appropriate sterilization methods. For example, one sterilization method comprises the steps of pre-filtration through a 3.0 microns filter followed by filtration through a 0.45 microns particle filter followed by steam or heat sterilization or sterile filtration through two redundant 0.2 microns membrane filters. The remaining steps in the process such as the homogenization and any solvent removal must then be conducted under sterile operating conditions. It is possible to completely avoid the use of steam or heat sterilization using the above described method of microprecipitation/homogenization since sterile filtration followed by aseptic operating procedures can be used.

The presuspension, final suspension, or dry powder form of the small particles can be sterilized by heat sterilization and irradiation regardless of the preparation method utilized.

In addition to the microprecipitation methods described above, any other known precipitation methods for preparing particles of active agent (and more preferably, small particles) in the art can be used in conjunction with the present invention.

The pharmaceutical compositions described herein may be administered by several routes of administration including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal routes of administration. The route of administration as well as the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

The pharmaceutical compositions described herein can optionally include one or more pharmaceutically acceptable excipients. Such pharmaceutically acceptable excipients are well known in the art and include, for example, salts, surfactant(s), water-soluble polymers, preservatives, antimicrobials, antioxidants, cryoprotectants, wetting agents, viscosity agents, tonicity modifying agents, levigating agents, absorption enhancers, penetration enhancers, pH modifying agents, muco-adhesive agents, coloring agents, flavoring agents, diluting agents, emulsifying agents, suspending agents, solvents, co-solvents, buffers, and combinations of these excipients.

The excipient included within the pharmaceutical compositions of the invention is chosen based on the expected route of administration of the composition in therapeutic applications. Accordingly, compositions designed for oral, lingual, sublingual, buccal and intrabuccal administration can be made without undue experimentation by means well known in the art, for example, with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Solid dosage forms, such as tablets, pills and capsules, may also contain one or more binding agents, filling agents, suspending agents, disintegrating agents, lubricants, sweetening agents, flavoring agents, preservatives, buffers, wetting agents, disintegrants, effervescent agents, and other excipients. Such excipients are known in the art. Examples of filling agents are lactose monohydrate, lactose anhydrous, and various starches. Examples of binding agents are various celluloses and cross-linked polyvinylpyrrolidone, microcrystalline cellulose, microcrystalline cellulose, and silicifized microcrystalline cellulose (SMCC). Suitable lubricants, including agents that act on the flowability of the powder to be compressed, are colloidal silicon dioxide, talc, stearic acid, magnesium stearate, calcium stearate, and silica gel. Examples of sweeteners are any natural or artificial sweetener, such as sucrose, xylitol, sodium saccharin, cyclamate, aspartame, and accsulfame K. Examples of flavoring agents are bubble gum flavor, fruit flavors, and the like. Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride. Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, lactose such as lactose monohydrate, lactose anhydrous, dibasic calcium phosphate, mannitol, starch, sorbitol, sucrose and glucose. Suitable disintegrants include corn starch, potato starch, maize starch, and modified starches, croscarmellose sodium, crosspovidone, sodium starch glycolate, and mixtures thereof. Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propyl parabens as preservatives, a dye and a flavoring such as cherry or orange flavor, and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administer to a wound site.

The compositions of the present invention can be administered parenterally such as, for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” is also meant to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

The pharmaceutical compositions described herein may be co-administered with one or more additional agents separately or in the same formulation. Such additional agents include, for example, anti-histamines, beta agonists (e.g., albuterol), antibiotics, anti-inflammatories (e.g. ibuprofen, prednisone (corticosteroid) or pentoxifylline), anti-fungals, (e.g. Amphotericin B, Fluconazole, Ketoconazol, and Itraconazol), steroids, decongestants, bronchodialators, and the like. The formulation may also contain preserving agents, solubilizing agents, chemical buffers, surfactants, emulsifiers, colorants, odorants and sweeteners.

The pharmaceutical composition described herein can be used to treat a patient suffering from a condition mediated by lipoxygenase and/or leukotriene activity. In one embodiment, the condition is mediated by 5- and/or 12-lipoxygenase activity. In another embodiment, the condition is an inflammatory condition.

Conditions mediated by lipoxygenase and/or leukotriene activity include, but are not limited to asthma, rheumatoid arthritis, gout, psoriases, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease (including, for example, ulcerative colitis and Crohn's disease), irritable bowel syndrome, cancer, tumors, respiratory syncytial virus, sepsis, endotoxin shock and myocardial infarction.

In one embodiment, the condition mediated by lipoxygenase and/or leuktoriene activity is an inflammatory condition. Inflammatory conditions include, but are not limited to, appendicitis, peptic, gastric or duodenal ulcers, peritonitis, pancreatitis, acute or ischemic colitis, diverticulitis, epiglottitis, achalasia, cholangitis, cholecystitis, hepatitis, inflammatory bowel disease (including, for example, Crohn's disease and ulcerative colitis), enteritis, Whipple's disease, asthma, chronic obstructive pulmonary disease, acute lung injury, ileus (including, for example, post-operative ileus), allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, pneumoultramicroscopic silicovolcanoconiosis, alvealitis, bronchiolitis, pharyngitis, pleurisy, sinusitis, influenza, respiratory syncytial virus, herpes, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thryoiditis, systemic lupus erythematosus, Goodpasture's syndrome, Behcet's syndrome, allograft rejection, graft-versus-host disease, Type I diabetes, ankylosing spondylitis, Berger's disease, Type II diabetes, Retier's syndrome, or Hodgkins disease.

In a further embodiment, the inflammatory condition is selected from the group consisting of rheumatoid arthritis, asthma, chronic obstructive pulmonary disease, acute lung injury, inflammatory bowel disease, allergy, organ ischemia, reperfusion injury, rhinitis, dermatitis, atherosclerosis, myocardial ischemia and adult respiratory distress syndrome.

The following is a description of examples of small particles of lipoxygenase inhibitor compounds and methods for making the same. The examples are for illustration purposes, and are not intended to limit the scope of the present invention.

### EXAMPLE 1

Preparation of a small-particle suspension having 3% (w/v) zileuton in an aqueous solution containing mPEG-DSPE, Poloxamer 188, glycerin and phosphate buffer is described below using a direct homogenization method.

Glycerin and sodium phosphate buffer were dissolved in distilled water to produce a 2.25% glycerin and lOmM phosphate buffer aqueous solution. mPEG-DSPE and Poloxamer 188 were then added so that each of these surfactants were present at 0.3% (w/v). The pH was adjusted to 7 with 1 N sodium hydroxide and/or hydrochloric acid solution. Zileuton was added to provide 3% (w/v) zileuton to form a pre-suspension.

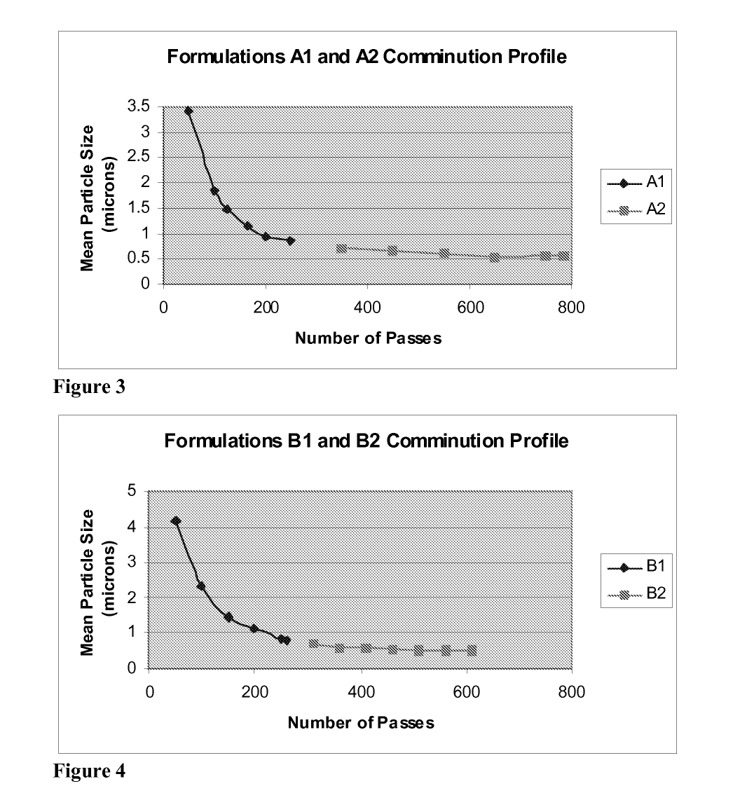

One aliquot of the pre-suspension was cycled through the piston-gap homogenizer for approximately 250 passes and a second aliquot was cycled through the homogenizer for approximately 800 passes to produce small-particle suspension formulations A1 and A2, respectively. The average particle size and the maximum particle size for 99% of the sample were determined by laser light scattering (Horiba LA-920). The results are as shown in FIG. 3.

### EXAMPLE 2

Preparation of a small-particle suspension having 3% (w/v) zileuton in an aqueous solution containing mPEG-DSPE, Poloxamer 188, glycerin and phosphate buffer is described below using a direct homogenization method.

Glycerin and sodium phosphate buffer were dissolved in distilled water to produce a 2.25% glycerin and IOmM phosphate buffer aqueous solution. mPEG-DSPE and Poloxamer 188 were then added so that each of these surfactants were present at 0.5% (w/v). The pH was adjusted to 7 with 1 N sodium hydroxide and/or hydrochloric acid solution. Zileuton was added to provide 3% (w/v) zileuton to form a pre-suspension.

One aliquot of the pre-suspension was cycled through the piston-gap homogenizer for approximately 260 passes and a second aliquot was cycled through the homogenizer for approximately 600 passes to produce small-particle suspension formulations B1 and B2, respectively. The average particle size and the maximum particle size for 99% of the sample were determined by laser light diffraction (Horiba LA-920). The results are as shown in FIG. 4.

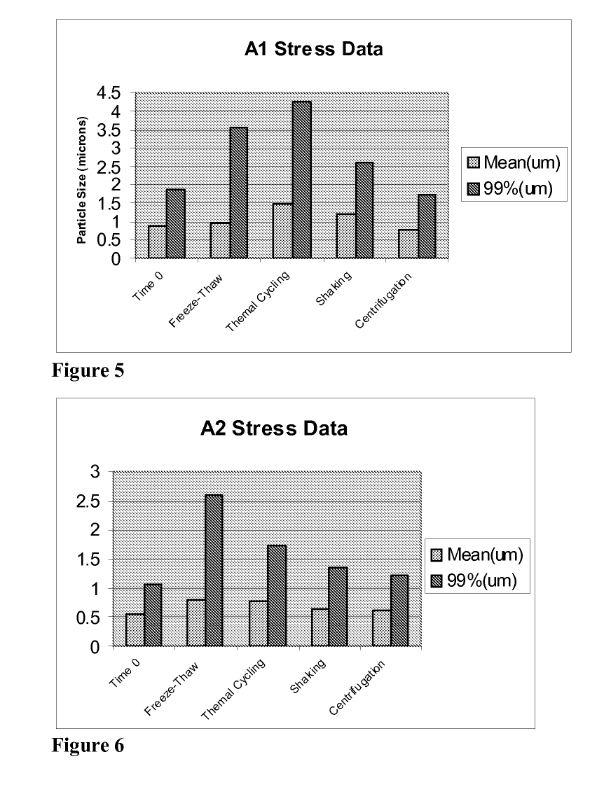

Formulations A1 and A2 were subjected to various stresses to determine their physical stability in terms of average particle size and the size for which 99% of the particles are smaller (volume weighted basis). One sample of each formulation was tested initially to serve as a baseline particle size determination. A second sample of each formulation was subjected to mechanical agitation (shaking). A third sample of each formulation was subjected to thermal cycling. A fourth sample of each formulation was subjected to centrifugation. The fifth sample was frozen and then thawed to room temperature. Particle size determinations were made by laser light diffraction (Horiba LA-920) on samples of each formulation as shown in FIGS. 5 and 6.

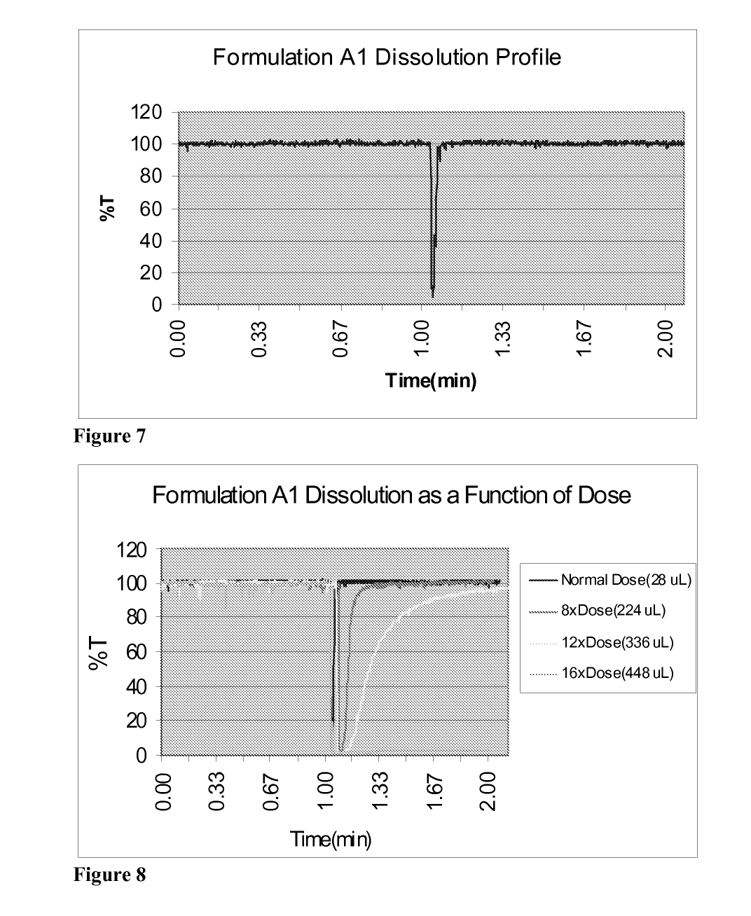

Dissolution results for formulation Al are shown in FIG. 7. Twenty-eight microliters of formulation A1 were injected into a measurement chamber containing 10-mL Sorensen's buffer and 5% albumin at 37° C. The time of the injection was recorded. Percent light transmittance was monitored versus time. The rapid dissolution of the small particles of zileuton under these circumstances should correspond to rapid release of the drug when injected intravenously providing peak plasma concentration of the drug relatively quickly.

Dissolution results for larger amounts or doses of formulation A1 are shown in FIG. 8. Twenty-eight microliters (1× dose), 224 microliters (8× dose), 336 microliters (12× dose), and 448 microliters (16× dose) of formulation A1 were injected into a dissolution chamber containing separate fresh aliquots of 10-mL Sorensen's buffer and 5% albumin and the time of the injection was recorded. Percent transmittance was monitored versus time.

### EXAMPLE 3

Preparation of a small-particle suspension having 3% (w/v) zileuton in an aqueous solution containing Lipoid E80, mPEG-DSPE, glycerin and phosphate buffer is described below using a direct homogenization method.

Glycerin and sodium phosphate buffer were dissolved in distilled water to produce a 2.25% glycerin and lOmM phosphate buffer aqueous solution. Lipoid E80 and mPEG-DSPE were then added so that Lipoid 80 was present at 1.5% (w/v) and mPEG-DSPE was present at 0.4% (w/v). The pH was adjusted to 7 with 1 N sodium hydroxide and/or hydrochloric acid solution. Zileuton was added to provide 3% (w/v) zileuton to form a pre-suspension.

The pre-suspension was cycled through the piston-gap homogenizer to produce small-particle suspension formulation C. The average particle size and the maximum particle size for 99% of the sample were determined by laser light diffraction (Horiba LA-920).

### EXAMPLE 4

Preparation of a small-particle suspension containing 3% (w/v) zileuton in an aqueous solution containing Tween80, Poloxamer 188, glycerin and phosphate buffer is described below using a direct homogenization method.

Glycerin and sodium phosphate buffer were dissolved in distilled water to produce a 2.25% glycerin and IOmM phosphate buffer aqueous solution. Tween 80 and Poloxamer 188 were then added so that Tween 80 was present at 0.25% (w/v) and Poloxamer 188 was present at 0.5% (w/v). The pH was adjusted to 7 with 1 N sodium hydroxide and/or hydrochloric acid solution. Zileuton was added to provide 3% (w/v) zileuton to form a pre-suspension.

The pre-suspension was cycled through the piston-gap homogenizer to produce small-particle suspension formulation D. The average particle size and the maximum particle size for 99% of the sample were determined by laser light diffraction (Horiba LA-920).

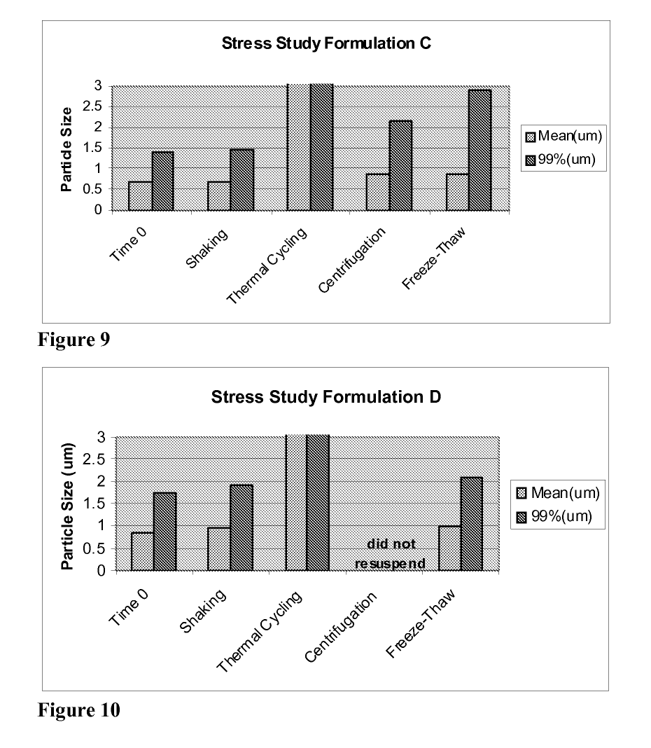

Formulations C and D were also subjected to various stresses to determine their physical stability in terms of average particle size and the size for which 99% of the particles are smaller (volume weighted basis). Particle size determinations were made by laser light diffraction (Horiba LA-920) on samples of each formulation as shown in FIGS. 9 and 10.

### EXAMPLE 5

Preparation of small-particle suspension having 3% zileuton (w/v) in an aqueous solution containing mPEG-DSPE, Poloxamer 188, sucrose and sodium phosphate buffer is described below using a direct homogenization method.

Sucrose and sodium phosphate buffer were dissolved in distilled water to produce a 9.25% sucrose and lOmM phosphate buffer aqueous solution. mPEG-DSPE and Poloxamer 188 were then added so that each of these surfactants were present at 0.5% (w/v). The pH was adjusted to 7 with 1 N sodium hydroxide and/or hydrochloric acid solution. Zileuton was added to provide 3% (w/v) zileuton to form a pre-suspension.

The pre-suspension was cycled through the piston-gap homogenizer for several passes to produce small-particle suspension formulation E.

### EXAMPLE 6

Preparation of small-particle suspension having 3% zileuton (w/v) in an aqueous solution containing mPEG-DSPE, Poloxamer 188, trehalose and sodium phosphate buffer is described below using a direct homogenization method.

Trehalose and sodium phosphate buffer were dissolved in distilled water to produce a 9.25% trehalose and 10 mM phosphate buffer aqueous solution. mPEG-DSPE and Poloxamer 188 were then added so that each of these surfactants were present at 0.5% (w/v). The pH was adjusted to 7 with 1 N sodium hydroxide and/or hydrochloric acid solution. Zileuton was added to provide 3% (w/v) zileuton to form a pre-suspension.

The pre-suspension was cycled through the piston-gap homogenizer for approximately 3 hours to produce small-particle suspension formulation F.

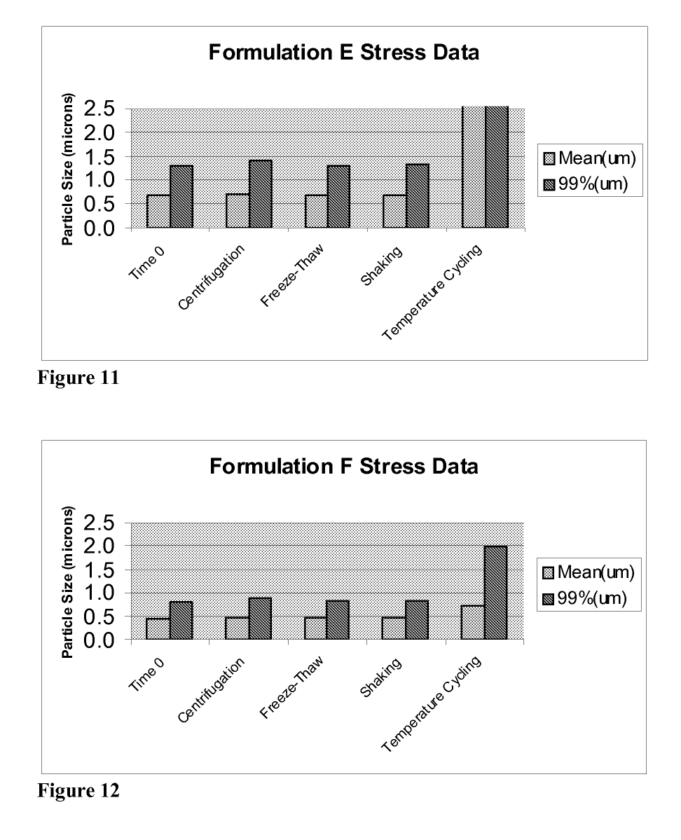

Formulations E and F were subjected to the stress conditions and procedures as discussed above. The average particle size and the size for which 99% of the particles are smaller (volume weighted basis) were determined by laser light diffraction (Horiba LA-920). The results are shown in FIGS. 11 and 12.

### EXAMPLE 7

Preparation of small-particle suspension having 3% zileuton (w/v) in an aqueous solution containing mPEG-DSPE, Poloxamer 188, trehalose and citrate buffer is described below using a direct homogenization method.

Trehalose, citric acid, and sodium citrate were dissolved in distilled water to produce a 9.25% (w/v) trehalose and lOmM citrate buffer aqueous solution. mPEG-DSPE and Poloxamer 188 were then added so that each of these surfactants were present at 0.5% (w/v). The pH was adjusted to 4 with 1 N sodium hydroxide and/or hydrochloric acid. Zileuton was added to provide 3% (w/v) zileuton to form a pre-suspension.

The pre-suspension was cycled through the piston-gap homogenizer multiple times to produce small-particle suspension formulation G.

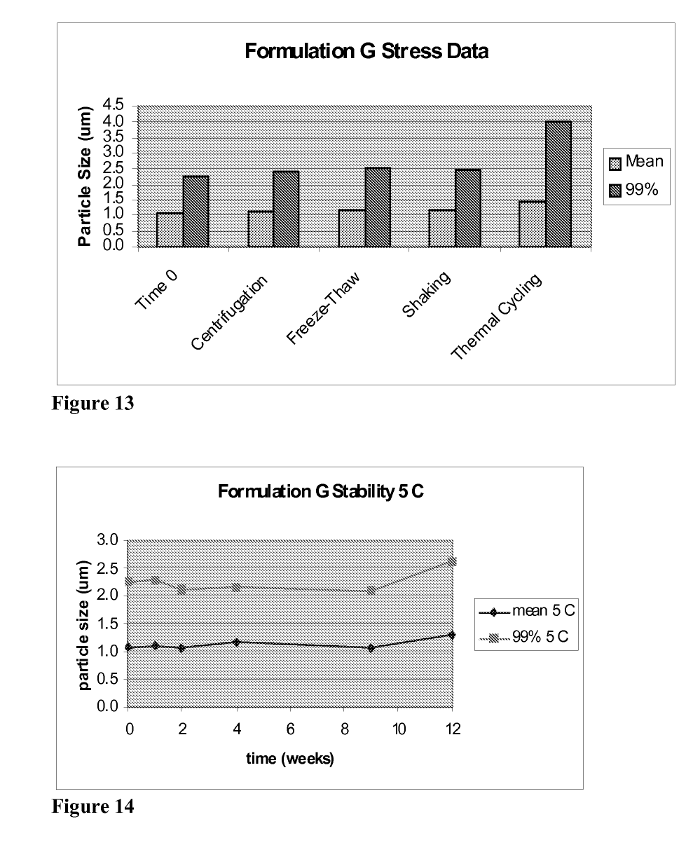

Formulation G was subjected to the stress conditions and procedures as discussed above. The average particle size and the size for which 99% of the particles are smaller (volume weighted basis) were determined by laser light diffraction (Horiba LA-920). The results are shown in FIG. 13.

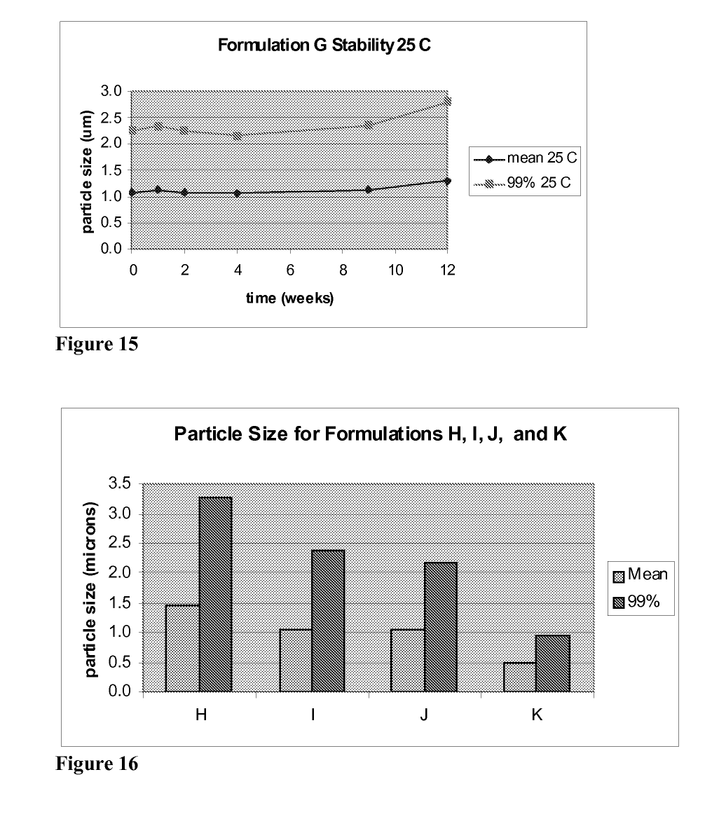

Samples of formulation G were stored at 5° C. and 25° C. for 12 weeks, and the average particle size and the size for which 99% of the particles are smaller (volume weighted basis) were determined by laser light diffraction (Horiba LA-920) at several time intervals. The results are shown in FIGS. 14 and 15.

### EXAMPLE 8

Preparation of a small particle suspension having 3% (w/v) zileuton in an aqueous solution containing deoxycholic acid sodium salt, Poloxamer 188, sucrose and phosphate buffer by the microprecipitation method is described below.

Sucrose and sodium phosphate buffer were dissolved in distilled water to produce a 9.25% (w/v) sucrose and 10 mM phosphate buffer aqueous solution. Deoxycholic acid sodium salt and Poloxamer 188 were then added so that each of these surfactants were present at 0.1% (w/v). The pH was adjusted to 7 with 1 N sodium hydroxide and/or hydrochloric acid solution. A second solution was prepared by dissolving zileuton in methanol. The two solutions were then combined to cause precipitation and formation of the pre-suspension.

The pre-suspension was cycled through the piston-gap homogenizer for several passes to produce small-particle suspension formulation H.

### EXAMPLE 9

Preparation of a small particle suspension having 3% (w/v) zileuton in an aqueous solution containing deoxycholic acid sodium salt, Poloxamer 188, trehalose and phosphate buffer by the microprecipitation method is described below.

Trehalose and sodium phosphate buffer were dissolved in distilled water to produce a 9.25% (w/v) trehalose and 10 mM phosphate buffer aqueous solution. Deoxycholic acid sodium salt and Poloxamer 188 were then added so that each of these surfactants was present at 0.1% (w/v). The pH was adjusted to 7 with 1 N sodium hydroxide and/or hydrochloric acid solution. A second solution was prepared by dissolving zileuton in methanol. The two solutions were then combined to cause precipitation and formation of the pre-suspension.

The pre-suspension was cycled through the piston-gap homogenizer for several passes to produce small-particle suspension formulation I.

### EXAMPLE 10

Preparation of a small particle suspension having 3% (w/v) zileuton in an aqueous solution containing mPEG-DSPE, Poloxamer 188, trehalose and phosphate buffer by the microprecipitation method using n-methyl pyrrolidinone (NMP) as the solvent is described below.

Trehalose and sodium phosphate buffer were dissolved in distilled water to produce a 9.25% (w/v) trehalose and lOmM phosphate buffer aqueous solution. mPEG-DSPE and Poloxamer 188 were then added so that each of these surfactants were present at 0.5% (w/v). The pH was adjusted to 7.5 with 1 N sodium hydroxide and/or hydrochloric acid solution. A second solution was prepared by dissolving zileuton in NMP. The two solutions were then combined to cause precipitation and formation of the pre-suspension.

The pre-suspension was cycled through the piston-gap homogenizer for several passes to produce small-particle suspension formulation J.

### EXAMPLE 11

Preparation of a small particle suspension having 3% (w/v) zileuton in an aqueous solution containing mPEG-DSPE, Poloxamer 188, trehalose and phosphate buffer by the microprecipitation method using methanol as the solvent is described below.

Trehalose and sodium phosphate buffer were dissolved in distilled water to produce a 9.25% (w/v) trehalose and 10 mM phosphate buffer aqueous solution. mPEG-DSPE and Poloxamer 188 were then added so that each of these surfactants were present at 0.5% (w/v). The pH was adjusted to 7.5 with 1 N sodium hydroxide and/or hydrochloric acid solution. A second solution was prepared by dissolving zileuton in methanol. The two solutions were then combined to cause precipitation and formation of the pre-suspension.

The pre-suspension was cycled through the piston-gap homogenizer for several passes to produce small-particle suspension formulation K.

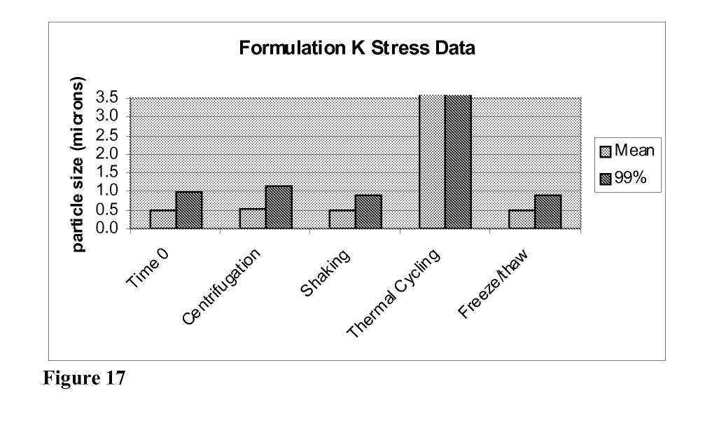

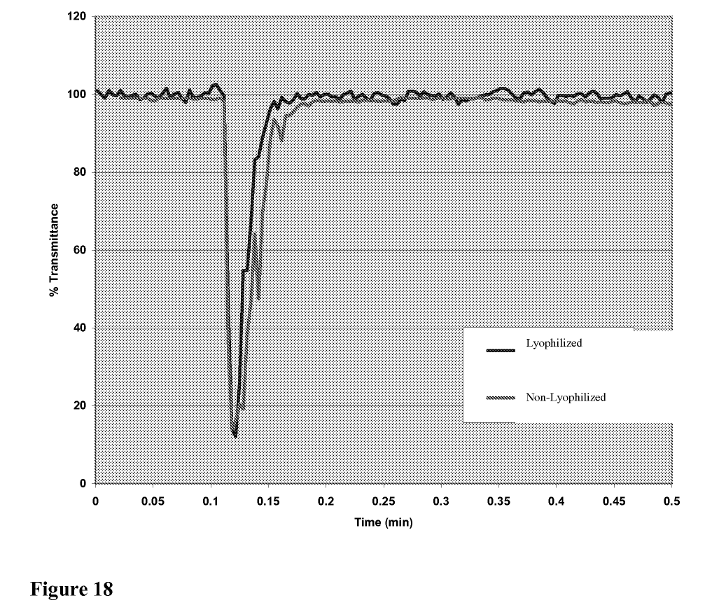

Particle size determinations were made by laser light diffraction (Horiba LA-920) on samples of formulations H, I, J, and K as shown in FIG. 16. In addition, samples of formulation K were subjected to the stress conditions and procedures as discussed above. The average particle size and the size for which 99% of the particles are smaller (volume weighted basis) were determined by laser light diffraction (Horiba LA-920). The results are shown in FIG. 17.

### EXAMPLE 12

Preparation of a small particle suspension having 3% (w/v) zileuton in an aqueous solution containing sodium deoxycholate, Poloxamer 188, sucrose and polyvinyl pyrrolidone by the microprecipitation method using methanol as the solvent is described below.

Sucrose was dissolved in distilled water to produce a 15% (w/v) sucrose aqueous solution. Sodium deoxycholate and Poloxamer 188 were then added so that each of these surfactants were present at 0.3% (w/v). The pH was adjusted to 7.5 with 1 N sodium hydroxide and/or hydrochloric acid solution. A second solution was prepared by dissolving zileuton in methanol. The two solutions were then combined to cause precipitation and formation of the pre-suspension.

The pre-suspension was cycled through the piston-gap homogenizer for several passes to produce a small-particle suspension formulation. The methanol was removed by centrifugation and a cryoprotectant, specifically polyvinyl pyrrolidone was added at about 0.5% (w/v). The zileuton concentration was adjusted to 3% (w/v) to produce small particle suspension Formulation L. 3.5 ml of Formulation L was placed in 10 ml tubing vials.

A batch of Formulation L vials was lyophilized for testing along with the non-lyophilized Formulation L. A typical lyophilization procedure was used consisting of freezing at −50° C., primary drying at −25° C. and 60 mTorr, and secondary drying at 30° C. and 60 mTorr. At time zero, the suspension (prior to lyophilization) is white and homogeneous, with a pH of approximately 7.3. Microscopic analysis indicated that the suspension consists of spherical, subspherical, and irregularly shaped particles less than 5 um in size; no drug particles or agglomerates greater than 10 um were observed.

Particle size results for both non-lyophilized and lyophilized Formulation L are summarized in Table C. The average particle size and the size for which 99% of the particles are smaller (volume weighted basis) were determined by laser light diffraction (Horiba LA-920). The suspension demonstrates an increase in particle size after lyophilization and reconstitution.

TABLE C

| | | |
|---|---|---|
| Sample | Mean (um) | 99%(um) |

Non-Lyophilized Suspension

| | | |
|---|---|---|
| 1 | 0.7400 | 1.666 |
| 2 | 0.6901 | 1.412 |
| 3 | 0.6845 | 1.378 |

Lyophilized Suspension (Reconstituted)

| | | |
|---|---|---|
| Lyo-1 | 1.1488 | 2.930 |
| Lyo-2 | 1.1411 | 2.738 |
| Lyo-3 | 1.2237 | 3.156 |

Potency testing was completed in triplicate using HPLC and the results are summarized in Table D. The levels of impurities/related substances for all samples were below the detectable limits of the HPLC method. The decrease in potency for the lyophilized samples may be attributable to losses due to the reconstitution method.

TABLE D
Time 0 Potency Analysis

| | |
|---|---|
| Sample | Potency (mg/mL) |

Non-Lyophilized Suspension

| | |
|---|---|
| 1 | 31.24 |
| 2 | 31.14 |
| 3 | 31.31 |

Lyophilized Suspension (Reconstituted)

| | |
|---|---|
| Lyo-1 | 29.04 |
| Lyo-2 | 28.86 |
| Lyo-3 | 29.37 |

Residual methanol concentration was determined by Gas Chromatography analysis. One sample was tested for the non-lyophilized suspension and one sample was tested for the reconstituted lyophilized suspension. The results are listed in Table F. The process of lyophilization may remove additional methanol from the suspension.

TABLE F
Residual Methanol Concentration

| Description | Methanol Conc. |
|---|---|
| Non-lyophilized suspension | 482 μg/mL |
| Lyophilized suspension, reconstituted | 93.5 μg/mL |

In order to characterize the dissolution rate of the nanosuspensions, a method involving the online monitoring of percent transmittance in a UV spectrophotometer was developed. The dissolution medium was a buffered solution containing albumin at pH 7.4. Each suspension sample was added to the dissolution medium contained in a spectrophotometer cell and the percent transmittance was recorded as a function of time at 400 nm. Testing was performed on both lyophilized and non-lyophilized Formulation L samples listed in Table J. The dissolution profiles are shown in FIG. 18. For both samples, the sharp decrease in the percent transmittance at approximately 0.1 min indicates the addition of the suspension to the dissolution medium. The percent transmittance then increases back to 100% as the suspension particles dissolve. It is observed that only a negligible difference can be seen in dissolution profiles and that both suspensions dissolved in approximately 3 seconds.

TABLE J
Dissolution Sample Information

| | Potency | Particle Size | |
|---|---|---|---|
| Description | (mg/mL) | Mean (um) | 99%(um) |
| Non-lyophilized suspension | 31.24 | 0.7190 | 1.509 |
| Lyophilized suspension, | 29.04 | 1.1835 | 2.920 |
| reconstituted | | | |

Samples of non-lyophilized and lyophilized suspension of Formulation L were stored at 5° C., 25° C., and 40° C. and tested at 4 week, 8 week and 12 week time frames.

Upon storage, the non-lyophilized samples showed sedimentation with a white to hazy supernatant. Aggregated particles were not visually observed. Many of the lyophilized cakes observed had slight cake shrinkage at the bottom of the vial, but all cakes maintained a white appearance with no significant collapse. The cakes reconstituted immediately upon addition of water for injection. For all samples observed at all intervals, the reconstituted suspension was white with no observable aggregates. pH testing was also conducted on the non-lyophilized suspension and the reconstituted lyophilized suspension and the results are listed in Tables L and M. The reconstituted lyophilized suspension samples showed less change in pH after storage relative to the initial pH.

TABLE L
pH of the Non-Lyophilized Suspension

| Sample | Temp ° C. | Initial | 4 Week | 8 Week | 12 Week |
|---|---|---|---|---|---|
| 1 | 5 | 7.249 | 7.43 | 7.44 | 7.48 |
| 2 | 5 | 7.330 | | | |
| 3 | 5 | 7.211 | | | |
| 1 | 25 | | 7.54 | 7.64 | 7.50 |
| 1 | 40 | | 7.56 | 7.80 | 7.75 |

TABLE M
pH of the Reconstituted Lyophilized Suspension

| | Sample ID | Inital | 4 week | 8 weeks | 12 weeks |
|---|---|---|---|---|---|
| 5° C. | 1 | 7.46 | 7.45 | 7.56 | 7.43 |
| | 2 | 7.54 | 7.49 | 7.39 | 7.45 |
| | 3 | 7.46 | 7.39 | 7.58 | 7.60 |
| 25° C. | 1 | | 7.52 | 7.64 | 7.54 |
| | 2 | | 7.50 | 7.58 | 7.41 |
| | 3 | | 7.40 | 7.53 | 7.44 |
| 40° C. | 1 | | 7.48 | 7.65 | 7.70 |
| | 2 | | 7.53 | 7.70 | 7.73 |
| | 3 | | 7.54 | 7.66 | 7.61 |

Tables N and O indicate the particle size results for the non-lyophilized suspension and the reconstituted lyophilized suspension. The non-lyophilized suspension shows a slight increase in particle size at 40° C. with time, whereas the reconstituted lyophilized suspension shows a larger increase in particle size upon storage at 25° C. and 40° C.

TABLE N
Particle Size Analysis of the Non-Lyophilized Suspension

| | Initial | | 4 Week | | 8 Week | | 12 Week | |
|---|---|---|---|---|---|---|---|---|
| | Mean | 99% | Mean | 99% | Mean | 99% | Mean | 99% |
| Sample | (μm) | (μm) | (μm) | (μm) | (μm) | (μm) | (μm) | (μm) |
| 1-5° C. | 0.7400 | 1.666 | 0.6532 | 1.349 | 0.5846 | 1.111 | 0.7193 | 1.582 |
| 2-5° C. | 0.6901 | 1.412 | | | | | | |
| 3-5° C. | 0.6845 | 1.378 | | | | | | |
| 1-25° C. | | | 0.7271 | 1.582 | 0.6204 | 1.163 | 0.8123 | 1.544 |
| 1-40° C. | | | 0.8743 | 2.280 | 0.9296 | 2.222 | 0.8889 | 2.059 |

TABLE O
Particle Size of the Reconstituted Lyophilized Suspension

| | | Time 0 | | 4 week | | 8 week | | 12 week | |
|---|---|---|---|---|---|---|---|---|---|
| | Sample | Mean | 99% | Mean | 99% | Mean | 99% | Mean | 99% |
| | ID | (μm) | (μm) | (μm) | (μm) | (μm) | (μm) | (μm) | (μm) |
| 5° C. | 1 | 1.1488 | 2.930 | 1.2215 | 3.974 | 1.3752 | 4.12 | 1.2084 | 2.874 |
| | 2 | 1.1411 | 2.738 | 1.0661 | 2.634 | 1.1769 | 2.955 | 1.1988 | 2.963 |
| | 3 | 1.2237 | 3.156 | 1.1111 | 2.823 | 1.1938 | 2.894 | 1.1848 | 2.948 |
| 25° C. | 1 | | | 1.3794 | 3.454 | 1.6679 | 4.911 | 1.5656 | 4.251 |
| | 2 | | | 1.4081 | 4.573 | 1.4498 | 3.830 | 1.5145 | 4.151 |
| | 3 | | | 1.5453 | 7.579 | 1.3867 | 3.748 | 1.4331 | 3.552 |
| 40° C. | 1 | | | 3.3651 | 9.042 | 1.7557 | 4.684 | 3.0828 | 7.126 |
| | 2 | | | 2.6449 | 8.254 | 2.0838 | 5.524 | 2.2774 | 5.631 |
| | 3 | | | 2.7704 | 8.304 | 2.1190 | 5.659 | 2.3775 | 5.808 |

Potency and related substances results for the non-lyophilized suspension are summarized in tables P and Q, respectively.

TABLE P
Potency (mg/mL) Results for Non-Lyophilized Suspension

| | | 5° C. | | 25° C. | | 40° C. | |
|---|---|---|---|---|---|---|---|
| Sample | | 8- | 12- | 8- | 12- | 8- | 12- |
| ID | Initial | week | week | week | week | week | week |
| 1 | 31.2 | 31.4 | 30.9 | 31.3 | 30.7 | 30.6 | 30.4 |

| | |
|---|---|
| 2 | 31.1 |
| 3 | 31.3 |

TABLE Q
Related Substances for Non-lyophilized Suspension²

| | | 5° C. | | 25° C. | | 40° C. | |
|---|---|---|---|---|---|---|---|
| | | 8- | 12- | 8- | 12- | 8- | 12- |
| Impurity | Initial | week | week | week | week | week | week |
| 1 | ND | ND | ND | ND | ND | 0.24 | 0.46 |
| 2 | ND | ND | ND | ND | 0.05 | 0.20 | 0.89 |

²results are %(w/w Formulation L)
ND = Not in Detectable Limits (Note: Detectable limit is 0.05%).

Potency and related substances results for the reconstituted lyophilized suspension are summarized in tables R and S, respectively. The results suggest that lyophilization may increase the chemical stability of the drug by decreasing the rate of drug degradation.

TABLE R
Potency (mg/mL) Results for the Reconstituted
Lyophilized Suspension

| | | 5° C. | | 25° C. | | 40° C. | |
|---|---|---|---|---|---|---|---|
| | | 8- | 12- | 8- | 12- | 8- | 12- |
| Sample | Initial | week | week | week | week | week | week |
| 1 | 29.0 | 28.9 | 29.5 | 24.7 | 27.7 | 28.6 | 29.4 |
| 2 | 28.9 | 24.5 | 30.0 | 28.3 | 28.4 | 29.2 | 29.6 |
| 3 | 29.4 | 28.8 | 33.1 | 27.1 | 30.2 | 28.6 | 28.8 |

TABLE S
Related Substances for the Reconstituted Lyophilized Suspension¹

| | | | 5° C. | | 25° C. | | 40° C. | |
|---|---|---|---|---|---|---|---|---|
| | Sam- | Ini- | 8- | 12- | 8- | 12- | 8- | 12- |
| Impurity | ple | tial | week | week | week | week | week | week |
| A66795-1 | 1 | ND | ND | ND | ND | ND | 0.07 | 0.06 |
| | 2 | ND | ND | ND | ND | ND | 0.06 | 0.07 |
| | 3 | ND | ND | ND | ND | ND | 0.06 | 0.06 |
| A66795-2 | 1 | ND | ND | ND | ND | ND | 0.13 | 0.13 |
| | 2 | ND | ND | ND | ND | ND | 0.12 | 0.16 |
| | 3 | ND | ND | ND | ND | ND | 0.13 | 0.14 |

¹results are %(w/w_{zileuton}).
ND = Not in Detectable Limits (Detectable Limit = 0.05%)

The dissolution of non-lyophilized suspension was tested per the method previously described. The dissolution medium was a buffered solution containing albumin at pH 7.4. Each sample was added to the dissolution medium contained in a spectrophotometer cell and the transmittance was recorded at 400 nm. The results indicate that the dissolution time after 12 weeks of storage did not increase for suspension stored at 5° C., 25° C., and 40° C. All samples dissolved in less than five seconds. Dissolution results for the reconstituted lyophilized suspension at an equivalent dose indicate no significant change in dissolution time after 12 weeks of storage at 5° C., 25° C., and 40° C. All samples showed complete dissolution in less than five seconds.

Water content by Karl Fischer titration was performed on three lyophilized samples at time zero and after storage at 5° C. for 12-weeks and the results are shown in Table W. During initial testing, sample 3 formed a precipitate while being tested, which may have contributed to the higher % RSD value. The higher average moisture content of the 12-week samples indicates that the lyophilized material is hygroscopic.

TABLE W
Karl Fischer Analysis of Lyophilized Samples

Average Moisture Content
(% w/w, n = 3)

| Sample | Initial | 12 week |
|---|---|---|
| 1 | 1.31% RSD 0.8% | 1.64% RSD 0.7% |
| 2 | 1.33% RSD 0.7% | 1.68% RSD 0.4% |
| 3 | 1.67% RSD 3.0% | 1.70% RSD 0.4% |

### EXAMPLE 13

Several preparations of a small particle suspension having 3% (w/v) zileuton in an aqueous solution were prepared by the microprecipitation method using methanol as the solvent. The formulations included a single or a combination of surfactants in addition to 15% sucrose. The formulations were unbuffered.

The formulations were prepared by dissolving sucrose in distilled water to produce a 15% (w/v) sucrose solution. The surfactants were then added so that each surfactant was present at the concentrations listed in Table X. The pH was adjusted to 8.0 with sodium hydroxide and/or hydrochloric acid solution. A second solution was prepared by dissolving zileuton in methanol. The two solutions were then combined to cause precipitation and formation of the pre-suspension containing approximately 3% (w/v) zileuton.

The pre-suspension was cycled through the piston-gap homogenizer for several passes to produce a small-particle suspension formulation. The methanol was removed by centrifiigation.

A batch of each formulation was lyophilized for testing along with the non-lyophilized suspensions. A typical lyophilization procedure was used consisting of freezing at −50° C., primary drying at −25° C. and 60 mTorr, and secondary drying at 30° C. and 60 mTorr.

The surfactant or combination of surfactants is identified along with particle size results for each non-lyophilized and lyophilized suspension in Table X. Post-lyophilization, testing was performed in duplicate (ie., two vials were reconstituted and tested).

TABLE X
Particle Size Data

| | | Pre Lyo | | Post Lyo | |
|---|---|---|---|---|---|
| Suspension | Surfactant | Mean | 99% | Mean | 99% |
| 1 | P188 | 10.2735 | 70.653 | 27.6989 | 169.849 |
| 2 | P188, Sodium | 8.4411 | 24.664 | 29.4597 | 224.077 |
| | Caprylate | | | | |
| 3 | 5% Albumin | 0.4349 | 0.948 | 19.0630 | 44.094 |
| | | | | 20.3334 | 45.843 |
| 4 | DMPC | 0.9260 | 2.474 | 10.7436 | 41.364 |
| | | | | 18.6608 | 41.168 |
| 5 | DMPC, DMPG | 1.1435 | 2.825 | 13.1264 | 42.623 |
| | | | | 22.8741 | 49.705 |
| 6 | DMPG, Tween80 | 0.9121 | 1.971 | 1.4102 | 5.784 |
| | | | | 15.3252 | 44.848 |
| 7 | 0.2% DMPG, | 0.6285 | 1.377 | 0.9701 | 2.806 |
| | 0.3% P188 | | | | |
| | | | | 0.9508 | 3.148 |
| 8 | 0.2% DMPG, | 1.1943 | 3.279 | 1.4254 | 6.328 |
| | 0.05% DPPA | | | | |
| | | | | 1.2557 | 4.084 |
| 9 | Tween80, DPPA | 0.7060 | 1.567 | 3.2418 | 9.849 |
| | | | | 5.0411 | 27.359 |

DMPC—dimyristoyl phosphatidylcholine; DMPG—dimyristoyl phosphatidylglycerol; DPPA—dipalmitoyl L-a-phosphatidic acid

In order to assess the effect of a cryoprotectant additional batches of the above fomulation were prepared with and without 0.2% (w/v) polyvinyl pyrrolidone. The poly vinyl pyrrolidone was added to the suspenions after the solvent removal and homogenization steps. The batches were lyophilized per the method described above and the particle size results are given in Table Y. Post-lyophilization, testing was performed in duplicate (i.e., two vials were reconstituted and tested).

TABLE Y
Particle Size Data

| Suspen- | | | Pre Lyo | | Post Lyo | |
|---|---|---|---|---|---|---|
| sion | Surfactant | % PVP | Mean | 99% | Mean | 99% |
| 1 | 0.2% DMPG, | 0% | 0.7086 | 1.694 | 3.3686 | 13.846 |
| | 0.3% P188 | | | | 3.2707 | 12.345 |
| 2 | | 0.2% | 0.6924 | 1.640 | 20.0295 | 37.781 |
| | | | | | 1.5243 | 6.707 |
| 3 | 0.2% DMPG, | 0% | 0.9419 | 2.258 | 2.1443 | 8.793 |
| | 0.05% DPPA | | | | 9.2706 | 49.502 |
| 4 | | 0.2% | 0.7697 | 1.842 | 0.6232 | 2.562 |
| | | | | | 0.9364 | 2.547 |

1. A pharmaceutical composition comprising an aqueous suspension of particles of a lipoxygenase inhibitor compound, wherein the particles have an effective average size of from about 10 nm to about 50 microns.

2. The pharmaceutical composition of claim 1 wherein the lipoxygenase inhibitor compound is selected from the group consisting of a 5-lipoxygenase inhibitor compound, a 12-lipoxygenase inhibitor and a compound that inhibits 5- and 12-lipoxygenase.

3. The pharmaceutical composition of claim 2, wherein the lipoxygenase inhibitor compound is selected from Formula (II): 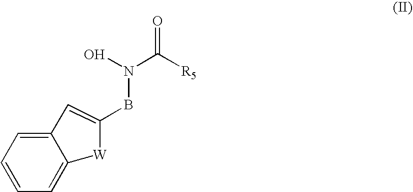
wherein R₅ is C1 or C2 alkyl or NR₆R₇, where R₆ and R₇ are independently selected from hydrogen and C1 or C2 alkyl; B is CH₂ or CHCH₃; and W is oxygen, sulfur, or nitrogen.

4. The pharmaceutical composition of claim 3 wherein the lipoxygenase inhibitor has the Formula (III):

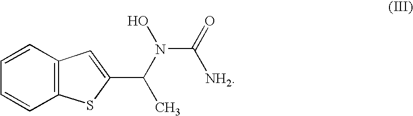

5. The pharmaceutical composition of claim 4 further comprising a pharmaceutically acceptable excipient.

6. The pharmaceutical composition of claim 4 wherein the lipoxygenase inhibitor is selected from the group consisting of ((±)-1-(1-benzo[b]thien-2-ylethyl)-1-hydroxyurea, the (−) isomer of 1-(1-benzo[b]thien-2-ylethyl)-1-hydroxyurea and the (+)-isomer of 1-(1-benzo[b]thien-2-ylethyl)-1-hydroxyurea.

7. The pharmaceutical composition of claim 4 further comprising at least one surfactant selected from the group consisting of ionic surfactants, non-ionic surfactants, zwitterionic surfactants, biologically derived surfactants, polymeric surfactants, amino-acid surfactants and derivatives of amino-acid surfactants.

8. The pharmaceutical composition of claim 7, wherein the nonionic surfactant is selected from the group consisting of polyoxyethylene fatty alcohol ethers, polyoxyethylene sorbitan fatty acid esters, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, p-isononylphenoxypoly(glycidol), decanoyl-N-methylglucamide, n-decyl-β-D-glucopyranoside, n-β-decyl-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside, nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

9. The pharmaceutical composition of claim 7, wherein the ionic surfactant is an anionic surfactant.

10. The pharmaceutical composition of claim 9, wherein the anionic surfactant is selected from the group consisting of alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, calcium stearate, phosphates, sodium dodecylsulfate, carboxymethylcellulose calcium, carboxymethylcellulose sodium, dioctylsulfosuccinate, dialkylesters of sodium sulfosuccinic acid, sodium lauryl sulfate, and a phospholipid.

11. The pharmaceutical composition of claim 10, wherein the phospholipid is selected from the group consisting of a phosphatide, a charged phospholipid, PEG-phospholipid, phosphatidylcholine, phosphatidylethanolamine, diacyl-glycero-phosphoethanolamine, dimyristoyl-glycero-phosphoethanolamine (DMPE), dipalmitoylglycerophosphoethanol-amine (DPPE), distearoylglycerophosphoethanolamine (DSPE), dioleolylglycerophosphoethanolamine (DOPE), phosphatidylserine, phosphatidylinositol, phosphatidylglycerol, phosphatidylinosine, phosphatidic acid, lysophospholipid, polyethylene glycolphospholipid conjugate, egg phospholipid, and soybean phospholipid.

12. The pharmaceutical composition of claim 7, wherein the ionic surfactant is a cationic surfactant.

13. The pharmaceutical composition of claim 12, wherein the cationic surfactant is selected from the group consisting of quaternary ammonium compounds, benzalkonium chloride, cetyltrimethylammonium bromide, chitosans, lauryldimethylbenzylammonium chloride, acyl carnitine hydrochlorides, alkyl pyridinium halides, cetyl pyridinium chloride, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15 trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT, tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, MIRAPOL, ALKAQUAT, alkyl pyridinium salts, amines, amine salts, imide azolinium salts, protonated quaternary acrylamides, methylated quaternary polymers, and cationic guar gum. benzalkonium chloride, dodecyl trimethyl ammonium bromide, triethanolamine, and poloxamines.

14. The pharmaceutical composition of claim 7, wherein the zwitterionic surfactant is selected from the group consisting of phosphatidylcholine, phosphatidylethanolamine, diacyl-glycero-phosphoethanolamine, dimyristoyl-glycero-phosphoethanolamine, dipalmitoyl-glycero-phosphoethanolamine, distearoyl-glycero-phosphoethanolamine, and dioleolyl-glycero-phosphoethanolamine.

15. The pharmaceutical composition of claim 7, further comprising a pH adjusting agent selected from the group consisting of sodium hydroxide, hydrochloric acid, tris buffer, mono-, di-, tricarboxylic acids and their salts, citrate buffer, phosphate buffer, acetate, lactate, tris(hydroxymethyl)aminomethane, aminosaccharide, mono-, di- and trialkylated amine, meglumine (N-methylglucosamine), succinate, benzoate, tartrate, carbonate and an amino acid.

16. The pharmaceutical composition of claim 15, further comprising an osmotic pressure adjusting agent selected from the group consisting of glycerin, an inorganic salt, monosaccharide, disaccharide, trisaccharide, and sugar alcohol.

17. The pharmaceutical composition of claim 16, wherein the lipoxygenase inhibitor compound is present in an amount from about 0.1 mg/ml to about 500 mg/ml.

18. The pharmaceutical composition of claim 17, wherein the lipoxygenase inhibitor compound is present in an amount from about 5.0 mg/ml to about 100 mg/ml.

19. The pharmaceutical composition of claim 18, wherein the lipoxygenase inhibitor compound is present in an amount from about 10 mg/ml to about 50 mg/ml.

20. The pharmaceutical composition of claim 19, wherein the particles have an effective average particle size of from about 50 nm to about 10 microns.

21. The pharmaceutical composition of claim 20, wherein the particles have an effective average particle size of from about 50 nm to about 2 microns.

22. The pharmaceutical composition of claim 19, wherein the surfactant is a polysorbate.

23. The pharmaceutical composition of claim 19, wherein the surfactant is a phospholipid.

24. The pharmaceutical composition of claim 19, wherein the surfactant is a polyoxyethylene-polypropylene block copolymer.

25. The pharmaceutical composition of claim 22, further including a second surfactant selected from the group consisting of ionic surfactants, non-ionic surfactants, anionic surfactants, zwitterionic surfactants, biologically derived surfactants, polymeric surfactants, amino-acids surfactants and derivatives of amino-acid surfactants.

26. The pharmaceutical composition of claim 23, further including a second surfactant selected from the group consisting of ionic surfactants, non-ionic surfactants, anionic surfactants, zwitterionic surfactants, biologically derived surfactants, polymeric surfactants, amino-acids surfactants and derivatives of amino-acid surfactants.

27. The pharmaceutical composition of claim 24, further including a second surfactant selected from the group consisting of ionic surfactants, non-ionic surfactants, anionic surfactants, zwitterionic surfactants, biologically derived surfactants, amino-acids surfactants and derivatives of amino-acid surfactants.

28. The pharmaceutical composition of claim 25, wherein the polysorbate is Tween 80 and the second surfactant is Poloxamer 188.

29. The pharmaceutical composition of claim 26, wherein the phospholipid is a PEG-DSPE and the second surfactant is Poloxamer 188.

30. The pharmaceutical composition of claim 26, wherein the phospholipid is a PEG-DSPE and the second surfactant is Lipoid E80.

31. The pharmaceutical composition of claim 26, wherein the phospholipid is dipalmitoyl L-a-phosphatidic acid and the second surfactant is dimyristoyl phosphatidylglycerol.

32. The pharmaceutical composition of claim 27, wherein the polyoxyethylene-polypropylene block copolymer is poloxamer 188 and the second surfactant is sodium deoxycholate.

33. The pharmaceutical composition of claim 27, wherein the polyoxyethylene-polypropylene block copolymer is poloxamer 188 and the second surfactant is dimyristoyl phosphatidylglycerol.

34. The pharmaceutical composition of claim 19, wherein the pharmaceutical composition is administered by a route selected from the group consisting of parenteral, oral, buccal, pulmonary, intravenous, intramuscular, subcutaneous, aural, rectal, vaginal, ophthalmic, intradermal, intraoccular, intracerebral, intralymphatic, intraarcticular, intrathecal and intraperitoneal.

35. The pharmaceutical composition of claim 32 wherein said aqueous suspension is dried.

36. The pharmaceutical composition of claim 35, wherein said aqueous suspension is dried by lyophilization, spray-drying or super-critical fluid extraction.

37. The pharmaceutical composition of claim 36, wherein said dried composition is formulated into a solid dosage form selected from the group consisting of tablets, capsules, lozenges, suppositories, coated tablets, ampoules, suppositories, delayed release formulations, controlled release formulations, extended release formulations, pulsatile release formulations, immediate release formulations, gastroretentive formulations, effervescent tablets, fast melt tablets, oral liquid and sprinkle formulations.

38. The pharmaceutical composition of claim 36, wherein said composition is formulated into a form consisting of the group consisting of patches, powder preparations which can be inhaled, compositions, creams, ointments and emulsions.

39. The pharmaceutical composition of claim 20 wherein, following an intravenous administration of the pharmaceutical composition, the particles rapidly dissolve such that a peak plasma concentration is reached within less than about 8 hours.

40. A method of treating a condition mediated by lipoxygenase activity and/or leukotriene in a mammal in need thereof by administering a pharmaceutical composition comprising an aqueous suspension of particles of a lipoxygenase inhibitor compound selected from the group consisting of a 5-lipoxygenase inhibitor compound, a 12-lipoxygenase inhibitor and a compound that inhibits 5- and 12-lipoxygenase, wherein the particles have an effective average size of from about 10 nm to about 50 microns.

41. The method of claim 40 wherein the condition is selected from the group consisting of asthma, rheumatoid arthritis, gout, psoriases, allergic rhinitis, respiratory distress syndrome, chronic obstructive pulmonary disease, acne, atopic dermatitis, atherosclerosis, aortic aneurysm, sickle cell disease, acute lung injury, ischemia/reperfusion injury, nasal polyposis, inflammatory bowel disease, irritable bowel syndrome, cancer, tumors, respiratory syncytial virus, sepsis, endotoxin shock and myocardial infarction.

42. The method of claim 40, wherein the condition is an inflammatory condition.

43. A method of making a pharmaceutical suspension comprising particles of a lipoxygenase inhibitor compound have an effective average size of from about 10 nm to about 50 microns by a precipitation method.

44. A method of making a pharmaceutical suspension comprising particles of a lipoxygenase inhibitor compound have an effective average size of from about 10 nm to about 50 microns by a microprecipitation method with energy addition.

45. A method of making a pharmaceutical suspension comprising particles of a lipoxygenase inhibitor compound have an effective average size of from about 10 nm to about 50 microns, the method comprising:
dissolving the lipoxygenase inhibitor compound in a water-miscible solvent to form a solution;
mixing the solution with the another solvent to define a pre-suspension; and
adding energy to the pre-suspension to form particles of the lipoxygenase inhibitor compound having an average effective particle size of from about 10 nm to about 50 microns.

46. The method of claim 45 wherein the lipoxygenase inhibitor compound is selected from the group consisting of a 5-lipoxygenase inhibitor compound, a 12-lipoxygenase inhibitor compound and a compound that inhibits 5- and 12-lipoxygenase.

47. The method of claim 46 wherein the lipoxygenase inhibitor compound is selected from Formula (II): 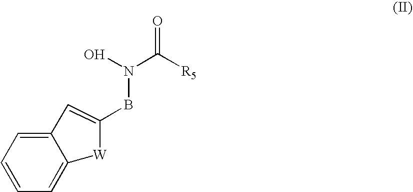
wherein R₅ is C₁ or C₂ alkyl or NR₆R₇, where R₆ and R₇ are independently selected from hydrogen and C₁ or C₂ alkyl; B is CH₂ or CHCH₃; and W is oxygen, sulfur, or nitrogen.

48. The method of claim 47 wherein the lipoxygenase inhibitor has the Formula (III):

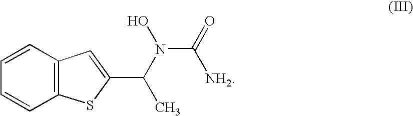

49. The method of claim 48, wherein at least one of the water-miscible solvent and the another solvent comprises at least one surfactant selected from the group consisting of an ionic surfactant, a non-ionic surfactant, a zwitterionic surfactant, a biologically derived surfactants, a polymeric surfactant, an amino-acids surfactant and a derivative of amino-acid surfactant.

50. The method of claim 49, wherein the nonionic surfactant is selected from the group consisting of polyoxyethylene fatty alcohol ethers, polyoxyethylene sorbitan fatty acid esters, polyoxyethylene fatty acid esters, sorbitan esters, glyceryl esters, glycerol monostearate, polyethylene glycols, polypropylene glycols, polypropylene glycol esters, cetyl alcohol, cetostearyl alcohol, stearyl alcohol, aryl alkyl polyether alcohols, polyoxyethylene-polyoxypropylene copolymers, poloxamers, poloxamines, methylcellulose, hydroxycellulose, hydroxymethylcellulose, hydroxypropylcellulose, hydroxypropylmethylcellulose, noncrystalline cellulose, polysaccharides, starch, starch derivatives, hydroxyethylstarch, polyvinyl alcohol, polyvinylpyrrolidone, triethanolamine stearate, amine oxides, dextran, glycerol, gum acacia, cholesterol, tragacanth, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, hydroxypropyl celluloses, hydroxypropyl methylcellulose, methylcellulose, hydroxyethylcellulose, hydroxypropylmethylcellulose phthalate, noncrystalline cellulose, polyvinyl alcohol, polyvinylpyrrolidone, 4-(1,1,3,3-tetramethylbutyl)phenol polymer with ethylene oxide and formaldehyde, poloxamers, alkyl aryl polyether sulfonates, mixtures of sucrose stearate and sucrose distearate, , p-isononylphenoxypoly(glycidol), decanoyl-N-methylglucamide, n-decyl-β-D-glucopyranoside, n-β-decyl-D-maltopyranoside, n-dodecyl-β-D-glucopyranoside, n-dodecyl-β-D-maltoside, heptanoyl-N-methylglucamide, n-heptyl-β-D-glucopyranoside, n-heptyl-β-D-thioglucoside, n-hexyl-β-D-glucopyranoside; nonanoyl-N-methylglucamide, n-nonyl-β-D-glucopyranoside, octanoyl-N-methylglucamide, n-octyl-β-D-glucopyranoside, octyl-β-D-thioglucopyranoside, PEG-cholesterol, PEG-cholesterol derivatives, PEG-vitamin A, PEG-vitamin E, and random copolymers of vinyl acetate and vinyl pyrrolidone.

51. The method of claim 49 where the ionic surfactant is an anionic surfactant.

52. The method of claim 51, wherein the anionic surfactant is selected from the group consisting of alkyl sulfonates, aryl sulfonates, alkyl phosphates, alkyl phosphonates, potassium laurate, sodium lauryl sulfate, sodium dodecylsulfate, alkyl polyoxyethylene sulfates, sodium alginate, dioctyl sodium sulfosuccinate, phosphatidic acid and their salts, sodium carboxymethylcellulose, bile acids and their salts, cholic acid, deoxycholic acid, glycocholic acid, taurocholic acid, and glycodeoxycholic acid, and calcium carboxymethylcellulose, stearic acid and its salts, calcium stearate, phosphates, sodium dodecylsulfate, carboxymethylcellulose calcium, carboxymethylcellulose sodium, dioctylsulfosuccinate, dialkylesters of sodium sulfosuccinic acid, sodium lauryl sulfate, and a phosphlipid.

53. The method of claim 52, wherein the phospholipid is selected from the group consisting of phosphatide, a charged phospholipid, PEG-phospholipid, phosphatidylcholine, phosphatidylethanolamine, diacyl-glycero-phosphoethanolamine, dimyristoyl-glycero-phosphoethanolamine (DMPE), dipalmitoylglycerophosphoethanolamine (DPPE), distearoylglycerophosphoethanolamine (DSPE), dioleolylglycerophosphoethanolamine (DOPE), phosphatidylserine, phosphatidylinositol, phosphatidylglycerol, phosphatidylinosine, phosphatidic acid, lysophospholipids, polyethylene glycol-phospholipid conjugates, egg phospholipid, and soybean phospholipid.

54. The method of claim 49, wherein the ionic surfactant is a cationic surfactant.

55. The method of claim 54, wherein the cationic surfactant is selected from the group consisting of quaternary ammonium compounds, benzalkonium chloride, cetyltrimethylammonium bromide, chitosans, lauryldimethylbenzylammonium chloride, acyl carnitine hydrochlorides, alkyl pyridinium halides, cetyl pyridinium chloride, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulfate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C12-18)dimethylbenzyl ammonium chloride, N-alkyl (C14-18)dimethyl-benzyl ammonium chloride, N-tetradecylidmethylbenzyl ammonium chloride monohydrate, dimethyl didecyl ammonium chloride, N-alkyl and (C12-14) dimethyl 1-napthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl(C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C12 trimethyl ammonium bromides, C15 trimethyl ammonium bromides, C17 trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT, tetrabutylammonium bromide, benzyl trimethylammonium bromide, choline esters, benzalkonium chloride, stearalkonium chloride, cetyl pyridinium bromide, cetyl pyridinium chloride, halide salts of quaternized polyoxyethylalkylamines, MIRAPOL, ALKAQUAT, alkyl pyridinium salts, amines, amine salts, imide azolinium salts, protonated quaternary acrylamides, methylated quaternary polymers, and cationic guar gum. benzalkonium chloride, dodecyl trimethyl ammonium bromide, triethanolamine, and poloxamines.

56. The method of claim 49, wherein the zwitterionic surfactant is selected from the group consisting of phosphatidylcholine, phosphatidylethanolamine, diacyl-glycero-phosphoethanolamine, dimyristoyl-glycero-phosphoethanolamine, dipalmitoyl-glycero-phosphoethanolamine, distearoyl-glycero-phosphoethanolamine, and dioleolyl-glycero-phosphoethanolamine.

57. The method of claim 49, wherein the another solvent includes a pH adjusting agent selected from the group consisting of sodium hydroxide, hydrochloric acid, tris buffer, a monocarboxylic acid, a dicarboxylic acid, a tricarboxylic acid and their salts, citrate buffer, phosphate buffer, acetate, lactate, tris(hydroxymethyl)aminomethane, aminosaccharides, mono-, di- and trialkylated amines, meglumine (N-methylglucosamine), and an amino acid.

58. The method of claim 57, wherein the another solvent includes an osmotic pressure adjusting agent selected from the group consisting of glycerin, inorganic salts, monosaccharides, disaccharides, trisaccharides, and sugar alcohols.

59. The method of claim 58, wherein the lipoxygenase inhibitor compound is present in an amount from about 1.0 mg/ml to about 200 mg/ml.

60. The method of claim 59, wherein the lipoxygenase inhibitor compound is present in an amount from about 5.0 mg/ml to about 100 mg/ml.

61. The method of claim 60, wherein the lipoxygenase inhibitor compound is present in an amount from about 10 mg/ml to about 50 mg/ml.

62. The method of claim 61, wherein the presuspension is passed through the piston-gap homogenizer to form a suspension having particles with an effective average particle size of less than about 10 microns.

63. The method of claim 62, wherein the presuspension is passed through the piston-gap homogenizer to form a suspension having particles with an effective average particle size of less than about 2 microns.

64. The method of claim 61, wherein the surfactant is a phospholipid.

65. The method of claim 61, wherein the surfactant is a polyoxyethylene-polypropylene block copolymer.

66. The method of claim 64, wherein at least one of the water-miscible solvent and the another solvent includes a second surfactant selected from the group consisting of ionic surfactants, non-ionic surfactants, anionic surfactants, zwitterionic surfactants, biologically derived surfactants, amino-acids surfactants and derivatives of amino-acid surfactants.

67. The method of claim 65, wherein at least one of the water-miscible solvent and the another solvent includes a second surfactant selected from the group consisting of ionic surfactants, non-ionic surfactants, anionic surfactants, zwitterionic surfactants, biologically derived surfactants, amino-acids surfactants and derivatives of amino-acid surfactants.

68. The method of claim 64, wherein the phospholipid is dimyristoyl phosphatidylglycerol and the second surfactant is Poloxamer 188.

69. The method of claim 64, wherein the phospholipid is dipalmitoyl L-a-phosphatidic acid the second surfactant is dimyristoyl phosphatidylglycerol.

70. The method of claim 65, wherein the polyoxyethylene-polypropylene block copolymer is Poloxamer 188 and the second surfactant is sodium deoxycholate.

71. A method of making a pharmaceutical composition comprising particles of a lipoxygenase inhibitor compound have an effective average size of from about 10 nm to about 50 microns by homogenization.

72. The method of claim 71 comprising the steps of:
adding a lipoxygenase inhibitor compound to an aqueous solution to form a presuspension; and
passing the presuspension through a piston-gap homogenizer at least one time to form a suspension.

73. The method of claim 72, wherein the lipoxygenase inhibitor compound is selected from the group consisting of a 5-lipoxygenase inhibitor compound, a 12-lipoxygenase inhibitor compound and a compound that inhibits 5- and 12-lipoxygenase.

74. The method of claim 73 wherein the lipoxygenase inhibitor compound is a 5-lipoxygenase inhibitor compound selected from Formula (II): 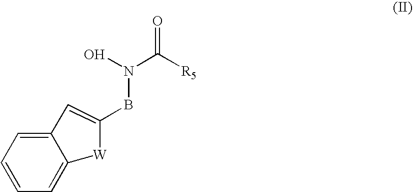
wherein R₅ is C₁ or C₂ alkyl or NR₆R₇, where R₆ and R₇ are independently selected from hydrogen and C₁ or C₂ alkyl; B is CH₂ or CHCH₃; and W is oxygen, sulfur, or nitrogen.

75. The method of claim 73 wherein the lipoxygenase inhibitor has the Formula (III) [chemical structure]
